(19) United States
(12) Patent Application Publication
(10) Pub. No.: US 2026/0224729 A1
(43) Pub. Date: Aug. 6, 2026

(54) ANTI-CD45 ANTIBODY-DRUG CONJUGATE PHARMACEUTICAL COMPOSITIONS

(71) Applicant: Regeneron Pharmaceuticals, Inc., Tarrytown, NY (US)

(72) Inventors: Amit Katiyar, Doylestown, PA (US); Kanyaphat Bunchongprasert, West Orange, NJ (US); Prashant Bhattarai, Attleboro, MA (US); Nicholas Yoder, Brookline, MA (US)

(21) Appl. No.: 19/573,704

(22) Filed: Mar. 20, 2026

Related U.S. Application Data
(63) Continuation of application No. PCT/US2024/047860, filed on Sep. 20, 2024.
(60) Provisional application No. 63/584,107, filed on Sep. 20, 2023.

(51) Int. Cl. A61K 47/68; A61K 47/02; A61K 47/20; A61K 47/22; A61K 47/26

(52) U.S. Cl. CPC A61K 47/6849; A61K 47/02; A61K 47/20; A61K 47/22; A61K 47/26; A61K 47/68035

(57) ABSTRACT

Disclosed are pharmaceutical compositions comprising an antibody drug conjugate comprising anti-CD45 antibody conjugated to an indolinobenzodiazepine (IGN).

### CROSS-REFERENCE TO RELATED APPLICATIONS

This application claims priority to U.S. Provisional Application No. 63/584,107, filed on Sep. 20, 2023, the entirety of which is incorporated herein by reference.

### FIELD OF INVENTION

The present disclosure relates generally to pharmaceutical compositions comprising an anti-CD45 antibody-drug conjugate (ADC).

### SEQUENCE LISTING

The instant application contains a Sequence Listing which has been submitted electronically in XML format and is hereby incorporated by reference in its entirety. Said XML copy, created on Sep. 20, 2024, is named V118216_2560WO_SL.XML and is 71,944 bytes in size.

### BACKGROUND

Antibody-drug conjugates (ADCs) comprise three essential components: an antibody that binds to a given target, a cytotoxin, and a linker that connects the antibody and the cytotoxin. Stabilizing an ADC in a pharmaceutical composition can be challenging. For example, each of the antibody, the linker, and the cytotoxin have unique physiochemical properties that require specific considerations with respect to a formulation. Identifying a formulation that provides stability for these complex molecules can be challenging (Duerr and Friess (2019) European J. of Pharmaceutics and Biopharmaceutics 139:168-176). Examples of challenges unique to ADCs include increased hydrophobicity versus the naked unconjugated antibody which can lead to increased aggregation. There remains a need for stable formulations of ADCs that can be used for therapeutic purposes.

### SUMMARY OF INVENTION

Disclosed herein are pharmaceutical compositions comprising an anti-CD45 antibody drug conjugate (ADC) comprising an anti-CD45 antibody and an indolinobenzodiazepine (IGN). The disclosed formulations are stable and suitable for administration to a human patient in need thereof.

Described herein are pharmaceutical compositions comprising an antibody-drug conjugate (ADC) comprising an anti-CD45 antibody and a sulfonated IGN cytotoxin, specifically DGN549. The disclosure provides stable pharmaceutical compositions of said ADC.

In one aspect, provided herein is a stable, pharmaceutical composition comprising an anti-CD45 antibody drug conjugate (ADC), a buffer, an antioxidant, a sugar, and a surfactant, wherein the ADC comprises an indolinobenzodiazepine (IGN) conjugated via a linker to an anti-CD45 antibody. The IGN may be conjugated via a cysteine residue in the Fc region of the anti-CD45 antibody.

In one embodiment, the antioxidant is sodium bisulfite, methionine, or both. In one embodiment, the pharmaceutical composition comprises about 25 to about 75 μM sodium bisulfite. In one embodiment, the pharmaceutical composition comprises about 50 μM sodium bisulfite. In one embodiment, the pharmaceutical composition comprises about 1 to about 5 mM methionine. In embodiment, the pharmaceutical composition comprises about 3 mM methionine. In certain embodiments, the pharmaceutical composition comprises methionine and sodium bisulfite, e.g., about 25 to about 75 μM sodium bisulfite and about 1 to about 5 mM methionine.

In one embodiment of the pharmaceutical composition, the surfactant is a polysorbate, e.g., polysorbate 20. In one embodiment, the pharmaceutical composition comprises about 0.01% polysorbate 20.

In another embodiment of the pharmaceutical composition, the buffer is histidine. In one embodiment, the pharmaceutical composition comprises a concentration of histidine suitable to buffer the composition to a pH of about 5.8 to 6.2. In another embodiment, the pharmaceutical composition comprises about 5 to about 15 mM histidine.

In one embodiment of the pharmaceutical composition, the sugar is sucrose. In certain embodiments, the pharmaceutical composition comprises about 10% sucrose.

In another aspect, provided herein is a stable, pharmaceutical composition comprising an anti-CD45 ADC, histidine, sodium bisulfite, sucrose, methionine and polysorbate 20, wherein the ADC comprises an IGN conjugated via a linker to an anti-CD45 antibody. In one embodiment, the pharmaceutical composition comprises about 0.01% polysorbate 20. The IGN may be conjugated via a cysteine residue in the Fc region of the anti-CD45 antibody.

In one embodiment, the pharmaceutical composition comprises about 25 to about 75 μM sodium bisulfite, about 5 to about 15 mM histidine, about 5% to 10% sucrose, and about 5 to 15 mM methionine. In one embodiment, the pharmaceutical composition comprises about 0.01% polysorbate 20.

In one embodiment, the pharmaceutical composition comprises about 10 mM histidine, about 50 uM bisulfite, 10% sucrose, 3 mM methionine, about 0.01% polysorbate 20.

In certain embodiments, the pharmaceutical composition of the invention has a pH of about 5.8 to about 6.2. In one embodiment, the pharmaceutical composition has a pH of about 6.

In one embodiment, the ADC has a toxin:antibody ratio of about 1.8 to 2.2. In another embodiment, the ADC has a toxin:antibody ratio of 2.

In one embodiment, the IGN has a formula

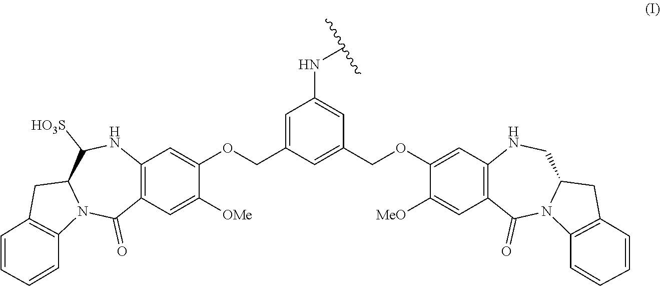

wherein the wavy line indicates the point of covalent attachment to the linker of the ADC.

In one embodiment, the IGN has a formula

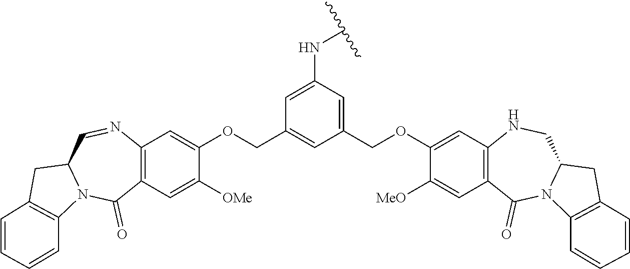 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wavy line indicates the point of covalent attachment to the linker of the ADC

In one embodiment, the pharmaceutical composition comprises a mixture anti-CD45 ADCs, wherein the mixture comprises an anti-CD45 antibody conjugated via a linker to an IGN as set forth in the following formulae:

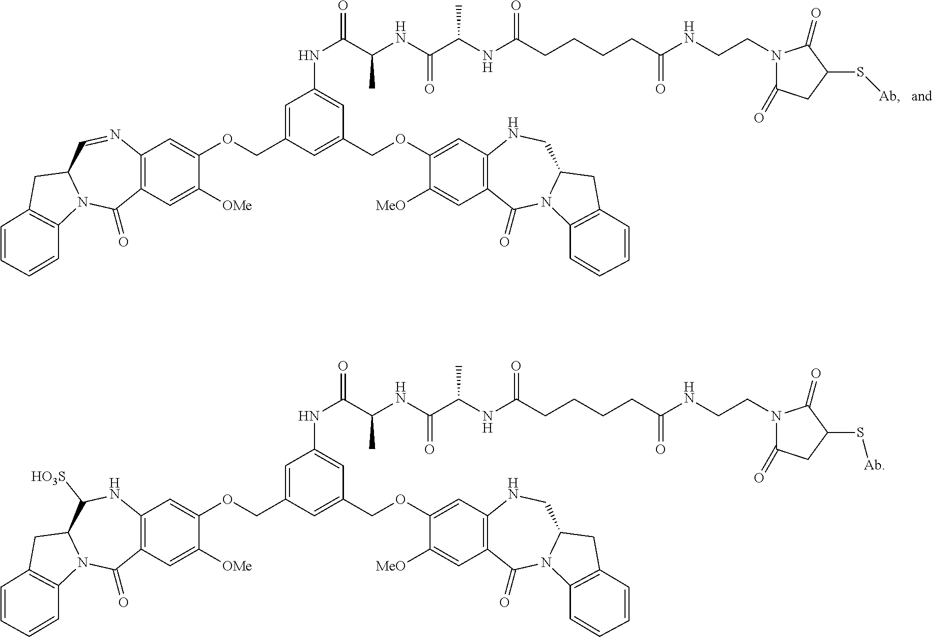

where Ab is the anti-CD45 antibody.

In some embodiments, the pharmaceutical composition is aqueous and comprises about 2 mg/mL of the ADC.

In some embodiments, the pharmaceutical composition is liquid or lyophilized. In some embodiments, the lyophilized composition is a powder or a cake.

In some embodiments, the ADC has a toxin:antibody ratio of about 1.8 to 2.2.

In one embodiment, the anti-CD45 antibody is conjugated to the linker via a cysteine residue within the Fc region of the heavy chain of the antibody, e.g., at amino acid position 265 of the Fc region (numbering is EU by Kabat).

In one embodiment, the antibody is an IgG1 isotype.

In one embodiment, the anti-CD45 antibody comprises a heavy chain variable region comprising a CDR1 domain comprising the amino acid sequence as set forth in SEQ ID NO: 2, a CDR2 domain comprising the amino acid sequence as set forth in SEQ ID NO: 3, and a CDR3 domain comprising the amino acid sequence as set forth in SEQ ID NO: 4; and comprises a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO: 8.

In another embodiment, the anti-CD45 antibody comprises a heavy chain variable region amino acid sequence as set forth in SEQ ID NO: 1 and a light chain variable region amino acid sequence as set forth in SEQ ID NO: 5.

In yet another embodiment, the anti-CD45 antibody comprises a heavy chain amino acid sequence as set forth in SEQ ID NO: 9 and a light chain amino acid sequence as set forth in SEQ ID NO: 10.

In one aspect, disclosed herein is a pharmaceutical composition comprising an antibody-drug conjugate (ADC), and a pharmaceutically acceptable carrier, wherein the ADC comprises an anti-CD45 antibody (Ab) conjugated to an indolinobenzodiazepine (IGN) via a linker, wherein the IGN has a formula:

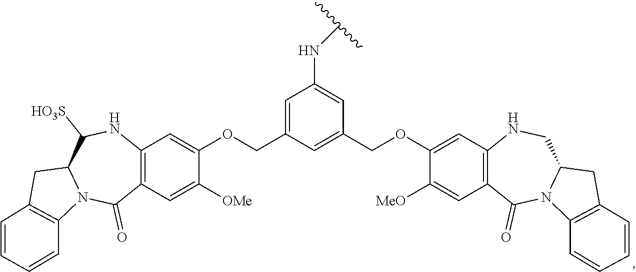

wherein the wavy line indicates the point of covalent attachment to the linker of the ADC, and wherein the anti-CD45 antibody comprises a heavy chain variable region comprising a CDR1 domain comprising the amino acid sequence as set forth in SEQ ID NO: 2, a CDR2 domain comprising the amino acid sequence as set forth in SEQ ID NO: 3, a CDR3 domain comprising the amino acid sequence as set forth in SEQ ID NO: 4; and comprises a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO:8, and is an IgG1 isotype. In some embodiments, the linker is

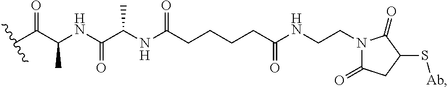

wherein the wavy line indicates the point of covalent attachment to the IGN and Ab is an anti-CD45 antibody.

In one aspect, disclosed herein is a pharmaceutical composition comprising an antibody-drug conjugate (ADC), wherein the ADC has a structure:

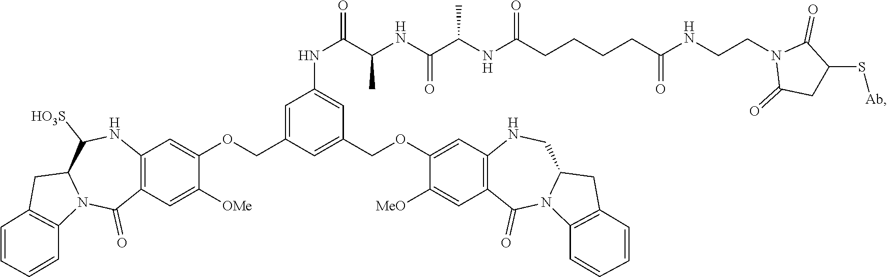

wherein Ab is an anti-CD45 antibody which is an IgG1 antibody and comprises a heavy chain variable region comprising a CDR1 domain comprising the amino acid sequence as set forth in SEQ ID NO: 2, a CDR2 domain comprising the amino acid sequence as set forth in SEQ ID NO: 3, a CDR3 domain comprising the amino acid sequence as set forth in SEQ ID NO: 4; and comprises a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO:8.

In some embodiments, the pharmaceutical compositions disclosed herein comprise histidine, methionine, a bisulfite salt, sucrose, and a polysorbate. In some embodiments, the polysorbate is polysorbate 20. In some embodiments, the pharmaceutical composition comprises 10 mM histidine. In some embodiments, the pharmaceutical composition comprises 3 mM methionine. In some embodiments, the pharmaceutical composition comprises 50 uM bisulfite salt. In some embodiments, the pharmaceutical composition comprises 10% sucrose (w/v). In some embodiments, the pharmaceutical composition comprises 0.01% polysorbate (w/v). In some embodiments, the pharmaceutical composition further comprises 5% dextrose. In some embodiments, the pharmaceutical composition has a pH of about 5.6 to 6.4. In some embodiments, the pharmaceutical composition has a pH of about 6.0. In some embodiments, the pharmaceutical composition comprises about 2 mg/mL of the ADC. In some embodiments, the pharmaceutical composition is liquid or lyophilized. In some embodiments, the lyophilized composition is a powder or a cake. In some embodiments, the ADC has a toxin:antibody ratio of about 1.8 to 2.2.

In some embodiments, the anti-CD45 antibody is conjugated to the linker via a cysteine residue at amino acid position 265 of the Fc region (numbering is EU by Kabat). In some embodiments, the anti-CD45 antibody comprises a heavy chain variable region amino acid sequence as set forth in SEQ ID NO: 1 and a light chain variable region amino acid sequence as set forth in SEQ ID NO: 5. In some embodiments, the anti-CD45 antibody comprises a heavy chain amino acid sequence as set forth in SEQ ID NO: 9 and a light chain amino acid sequence as set forth in SEQ ID NO: 10.

In one embodiment, the ADC described herein comprises an anti-CD45 antibody defined by the CDR, variable, or heavy and light chain amino acid sequences set forth in Table 10. IN another embodiment, the ADC described herein comprises an anti-CD45 antibody defined by amino acid sequences as described in US Patent Publication No. US-2024-0254251, which is incorporated by reference herein.

### BRIEF DESCRIPTION OF THE DRAWINGS

FIGS. 1A-1C graphically depict the results of a size-exclusion chromatography assay to determine the stability, specifically aggregation, of an anti-CD45 ADC following exposure to stress conditions. FIG. 1A depicts the percent of monomer (% monomer) observed across all conditions tested for the four formulations (F01 at 2 mg/ml (also called F1b) and 0.5 mg/ml (also called F1a), F2, and F3). FIG. 1B depicts the sum of the percent high molecular weight (% HMW) and percent low molecular weight (% LMW) species observed across all conditions tested for the four formulations (F01 at 2 mg/ml (also called F1b) and 0.5 mg/ml (also called F1a), F2, and F3). FIG. 1C provides the sum of the % HMW and % LMW species observed across conditions for the four formulations (F01 at 2 mg/ml (also called F1b) and 0.5 mg/ml (also called F1a), F2, and F3) tested, with the 40° C. condition excluded.

FIGS. 2A-2C graphically depict the results of a capillary electrophoresis sodium dodecyl sulfate (CE-SDS) assay to determine the stability of an anti-CD45 ADC following exposure to stress conditions. FIG. 2A depicts the percent purity (% purity) observed across all conditions tested. FIG. 2B depicts the percent impurity (% impurity) observed across all conditions tested. FIG. 2C provides the % impurity observed across conditions tested with the 40° C. condition excluded.

FIG. 3 depicts sulfonated DGN549-C and DGN549-C (DGN549-C describes DGN549 with a protease-cleavable linker which can be conjugated to a cysteine residue in the heavy chain of an antibody).

FIG. 4 graphically depicts the number of total particles between ≥1.0 and <100 μm determined for the antibody and the ADC. FCG1076 DS is the ADC.

FIGS. 5A and 5B provide s SEC overlays of an antibody sample (A) and an ADC sample (B) at pH 3.5 over 3 days.

FIG. 6 graphically depicts the % main peak as determined by SEC for the antibody (top line) and the ADC (lower line).

FIG. 7 graphically depicts a comparison of the antibody and the ADC charge heterogeneity % main peak purity across 3 days. The ADC showed a faster rate of degradation.

FIG. 8 provides a reaction scheme for chemical conjugation of an anti-CD45 antibody via a succinimide linkage to DGN549-C (DGN549 with a protease-cleavable linker).

### DETAILED DESCRIPTION

In order that the present disclosure may be more readily understood, certain terms are first defined.

### Definitions

As used herein, the term “about” refers to a value that is within 5% above or below the value being described. For example, the term “about 100 nM” indicates a range of 95-105 nM.

The terms “pharmaceutical formulation” or “pharmaceutical composition” are used interchangeably herein and refer to preparations comprising an active ingredient, such as an ADC, that are in such a form as to permit the biological activity of the active ingredient(s) to be effective and, therefore, may be administered to a subject for therapeutic use.

The term “stable”, as used herein, refers to a pharmaceutical formulation in which the ADC retains its physical and chemical integrity for a sufficient period of time to be useful for the purposes detailed herein (e.g., therapeutic or prophylactic administration to a subject). Preferably, the pharmaceutical formulations described herein are stable.

As used herein, a “diluent” refers to a pharmaceutically acceptable (i.e., safe and non-toxic for human administration) liquid solution useful in preparing an aqueous or reconstituted pharmaceutical composition. Exemplary diluents include sterile water, pH buffered solutions, sterile saline, and/or dextrose solution.

The term “buffer” refers to a compound that resists changes in pH by the action of its acid-base conjugate components when in solution, i.e., can maintain the pH value of a solution. In some embodiments, the pharmaceutical compositions disclosed herein comprise histidine buffer. In some embodiments, the pharmaceutical composition has a pH of about 5.6 to about 6.4. In some embodiments, the pharmaceutical composition has a pH of about 6.0.

The term “surfactant” as used herein is a compound that is typically used in pharmaceutical formulations to prevent drug adsorption to surfaces and or aggregation. Surfactants are amphiphilic, which means they are usually composed of both hydrophilic and hydrophobic or lipophilic groups, thus being capable of forming micelles or similar self-assembled structures in aqueous solutions. Known surfactants for use in pharmaceutical compositions include glycerol benzethonium chloride, cetrimide, sodium docusate, phospholipids, polyethylene alkyl ethers, sodium lauryl sulfate and tricaprylin (anionic surfactants); benzalkonium chloride, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl 15 hydroxystearate, polyoxylglycerides, polysorbates, propylene glycol dilaurate, propylene glycol monolaurate, sorbitan esters sucrose palmitate, sucrose stearate, tricaprylin and TPGS (Nonionic and zwitterionic surfactants). In one embodiment, a pharmaceutical composition described herein comprises a surfactant which is a polysorbate.

The term “sugar” as used herein includes sugars and sugar alcohols/polyols. Sugars can be referred to monosaccharides, disaccharides, and polysaccharides. Examples of sugars include, but are not limited to, sucrose, trehalose, glucose, dextrose, raffinose and others. Examples of sugar alcohols or polyols include, but are not limited to, mannitol, sorbitol, and others.

The term “antioxidant” refers to an agent that inhibits the oxidation of other molecules and is not part of buffer component. Examples of antioxidants herein include sodium bisulfite, citrate, lipoic acid, uric acid, glutathione, tocopherol, carotene, lycopene, cysteine, methionine, phosphonate compounds, e.g., etidronic acid, desferoxamine and malate.

As used herein, the term “conjugate”, “cytotoxin-linker conjugate”, “antibody drug conjugate” or “ADC” refers to an antibody which is linked to a cytotoxin via a linker. The terms are, used interchangeably throughout in reference to such a molecule. In one embodiment, an ADC is formed by the chemical bonding of a reactive functional group of one molecule, such as an antibody or antigen-binding fragment thereof, with an appropriately reactive functional group of another molecule, such as a cytotoxin described herein.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), and antibody fragments so long as they exhibit the desired antigen-binding activity. Preferably the antibody is a monoclonal antibody.

Generally, antibodies comprise heavy and light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

An “intact” or “full length” antibody, as used herein, refers to an antibody having two heavy (H) chain polypeptides and two light (L) chain polypeptides interconnected by disulfide bonds. In certain embodiments, a toxin can be conjugated to an intact anti-CD45 antibody having heavy and/or light chain amino acid sequences described herein.

The terms “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the entire second constant domain CH2 (residues at EU positions 231-340 of IgG1) and the third constant domain CH3 (residues at EU positions 341-447 of human IgG1). As used herein, the Fc domain includes the “lower hinge region” (residues at EU positions 233-239 of IgG1).

Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or Clq as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary.

The terms “Fc gamma receptor” or “Fc gamma R” as used herein refer to any member of the family of proteins that bind the IgG antibody Fc region and are encoded by the FcgammaR genes. In humans this family includes but is not limited to FcgammaRI (CD64), including isoforms FcgammaRIa, FcgammaRIb, and FcgammaRIc; FcgammaRII (CD32), including isoforms FcgammaRIIa (including allotypes H131 and R131), FcgammaRIIb (including FcgammaRIIb-1 and FcgammaRIIb-2), and FcgammaRIIc; and FcgammaRIII (CD16), including isoforms FcgammaRIIIa (including allotypes V158 and F158) and FcgammaRIIIb (including allotypes FcgammaRIIIb-NA1 and FcgammaRIIIb-NA2). An FcgammaR can be from any organism, including but not limited to humans, mice, rats, rabbits, and monkeys. Mouse FcgammaRs include but are not limited to FcgammaRI (CD64), FcgammaRII (CD32), FcgammaRIII (CD16), and FcgammaRIII-2 (CD16-2).

As used herein, the terms “condition” and “conditioning” refer to processes by which a patient is prepared for receipt of a transplant, e.g., a transplant containing hematopoietic stem cells and/or genetically-modified cells. Such procedures promote the engraftment of, e.g., a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of an ADC comprising an antibody or antigen-binding fragment thereof capable of binding CD45 expressed by hematopoietic stem cells. As described herein, the antibody may be covalently conjugated to a cytotoxin so as to form a drug-antibody conjugate. Administration of an ADC capable of binding the foregoing antigen to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

As used herein, the term “human antibody” is intended to include antibodies having variable regions derived from human germline immunoglobulin sequences. In embodiments in which a human antibody contains a constant region, the constant region can likewise be derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes.

As used herein, the term “allogeneic”, in the context of transplantation, is used to define a transplant (e.g., cells, tissue or an organ transplant) that is transplanted from a donor to a recipient, wherein the recipient is a different individual of the same species, relative to the donor.

As used herein, the term “autologous”, in the context of transplantation, refers to a transplant where the donor and recipient are the same individual, i.e., the same subject.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B cells and T cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short-term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin− (negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the terms “subject” and “patient” refer to an organism, such as a human, that receives treatment for a particular disease or condition as described herein. In some embodiments, the subject or patient referenced in the methods provided herein is a human subject.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein “to treat” or “treatment”, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. For example, treatment can refer to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeloblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD45+ leukemic cells) or autoimmune cells (e.g., CD45+ autoimmune lymphocytes, such as a CD45+ T-cell that expresses a T-cell receptor that cross-reacts with a self antigen). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from any disease or disorder disclosed herein. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues.

As used herein, the singular form “a,” “an,” and “the” comprises plural references unless indicated otherwise.

As used herein, where the term “about” is used, it is also contemplated that the recited number is considered as well, e.g., “about 6” includes 6 without variation as defined by the term “about” herein.

It is understood that embodiments, aspects and variations described herein also include “consisting” and/or “consisting essentially of” embodiments, aspects and variations.

As used herein, the phrase “consisting essentially of” when used in reference to a formulation, means that the formulation necessarily includes the list of recited elements (ADC and excipients) and is open to unlisted ingredients that do not materially affect the basic nature or stability of the formulation. Thus, other ingredients other than those expressly listed may be present, but such ingredients are found only in trace amounts or in amounts otherwise low enough that the fundamental characteristics of the formulation including protein concentration, viscosity, thermal stability, osmolality, and pH are unchanged.

### Pharmaceutical Compositions

Provided herein are stable, pharmaceutical compositions comprising an anti-CD45 antibody drug conjugate (ADC) comprising an anti-CD45 antibody conjugated via a linker to an indolinobenzodiazepine (IGN). In particular, the combination of a buffer, an antioxidant, a sugar, and a surfactant provides stability for an anti-CD45 ADC as disclosed herein.

The ADC pharmaceutical composition disclosed herein is stabilized, at least in part, by the presence of an antioxidant. In one embodiment, the antioxidant is sodium bisulfite, methionine, or both. In one embodiment, the pharmaceutical composition comprises about 25 to about 75 μM sodium bisulfite, about 30 to about 70 μM sodium bisulfite, about 35 to about 65 μM sodium bisulfite, about 40 to about 60 μM sodium bisulfite, about 45 to about 55 μM sodium bisulfite, or about 50 μM sodium bisulfite. The pharmaceutical composition can include, either as an alternative or in combination with sodium bisulfite, methionine as an antioxidant. In one embodiment, the pharmaceutical composition comprises about 1 to about 5 mM methionine, about 1.5 to about 4.5 mM methionine, about 2 to about 4 mM methionine, about 2.5 to about 3.5 mM methionine, or about 3 mM methionine. Ranges based on combinations of numbers recited in the foregoing ranges are also contemplated in addition to those recited, e.g., an amount sodium bisulfite which is 25 to 60 μM.

The ADC pharmaceutical compositions described herein may comprise a sugar. In some embodiments, the sugar is sucrose, a disaccharide consisting of glucose and fructose subunits. The ADC pharmaceutical composition may include an amount of sucrose which is about 5% to about 15% sucrose, about 6% to about 14% sucrose, about 7% to about 13% sucrose, about 8% to about 12% sucrose, about 9% to about 11% sucrose, or about 10% sucrose.

In some embodiments, the sugar is dextrose (i.e., D-glucose).

The pharmaceutical compositions described herein may comprise a surfactant. The term “surfactant” refers generally to organic substances that are amphipathic in nature, that is they comprise both a hydrophobic region and a hydrophilic region. In some embodiments, the surfactant is a nonionic surfactant, such as polysorbate. In some embodiments, the polysorbate is polysorbate-20 (also known as Tween™ 20).

In one embodiment, the ADC pharmaceutical composition comprises about 0.005% polysorbate 20 to about 0.015% polysorbate 20, about 0.006% polysorbate 20 to about 0.014% polysorbate 20, about 0.007% polysorbate 20 to about 0.013% polysorbate 20, about 0.008% polysorbate 20 to about 0.012% polysorbate 20, about 0.009% polysorbate 20 to about 0.011% polysorbate 20, or about 0.01% polysorbate 20. In one embodiment, the pharmaceutical composition comprises about 0.02% polysorbate 20 to about 1% polysorbate 20, about 0.04% polysorbate 20 to about 0.8% polysorbate 20, about 0.06% polysorbate 20 to about 0.6% polysorbate 20, about 0.08% polysorbate 20 to about 0.4% polysorbate 20, or about 0.1% polysorbate 20 to about 0.2% polysorbate 20. Ranges based on combinations of numbers recited in the foregoing ranges are also contemplated in addition to those recited, e.g., an amount of polysorbate 20 which is 0.015% to 0.4%.

As disclosed herein, the stability of a pharmaceutical composition comprising the anti-CD45 ADC is impacted by pH. Thus, the pharmaceutical composition comprises a buffer that helps to stabilize the pH of the formulation. In certain embodiments, the pH of a pharmaceutical composition disclosed herein is about 5 to about 7, about 5.2 to about 6.8, about 5.4 to about 6.6, 5.6 to about 6.4, about 5.7 to about 6.3, about 5.8 to about 6.2, about 5.9 to about 6.1, or about 6. Ranges based on combinations of numbers recited in the foregoing pH ranges are also contemplated in addition to those recited, e.g., a pH of 5 to 6.4.

In one embodiment, histidine is included as a buffer in the pharmaceutical composition of the invention. Amounts of histidine suitable to maintain a pH within the foregoing ranges can be used. In one embodiment, the pharmaceutical composition comprises about 1 to about 30 mM histidine, about 1 to about 28 mM histidine, about 1 to about 26 mM histidine, about 1 to about 24 mM histidine, about 1 to about 22 mM histidine, about 1 to about 20 mM histidine, about 1 to about 18 mM histidine, about 2 to about 18 mM histidine, about 3 to about 18 mM histidine, about 4 to about 18 mM histidine, 5 to about 15 mM histidine, about 6 to about 14 mM histidine, about 7 to about 13 mM histidine, about 8 to about 12 mM histidine, about 9 to about 11 mM histidine, or about 10 mM histidine. Ranges based on combinations of numbers recited in the foregoing histidine molar ranges are also contemplated in addition to those recited, e.g., a 3 to 12 mM histidine.

The pharmaceutical compositions described herein confer stability to an anti-CD45 ADC such that it retains both its physiochemical stability and biological activity during storage, including until administration to a subject. An anti-CD45 ADC retains its physiochemical stability if it shows minimal signs of aggregation, precipitation, and/or denaturation as measured by methods known in the art. An anti-CD45 ADC retains its biological activity if the pharmaceutical composition does not impact the intended purpose of the anti-CD45 ADC (i.e., retains its use for the therapeutic uses described herein). For example, an anti-CD45 ADC retains its biological activity if it retains the ability to deplete CD45+ cells in a human subject.

In one aspect, disclosed herein are pharmaceutical compositions comprising an anti-CD45 ADC (wherein the anti-CD45 antibody is conjugated to one or more sulfonated DGN549 molecules) that maintains the biological activity of the ADC.

The pharmaceutical compositions described herein may be aqueous, a lyophilized powder, or a lyophilized cake. As used herein, the term “lyophilized cake” refers to a lyophilized product that maintains the size and shape of the frozen liquid product.

In one aspect, the pharmaceutical composition comprises an anti-CD45 ADC, a sugar, a buffer, and a surfactant. In some embodiments, the pharmaceutical composition has a pH of about 5.6 to about 6.4.

In some embodiments, the pharmaceutical composition comprises an anti-CD45 ADC, sucrose, histidine, methionine, a bisulfite salt (e.g., sodium bisulfite), and a polysorbate. In some embodiments, the polysorbate is polysorbate 20. In some embodiments, the pharmaceutical composition has a pH of about 6.0.

In some embodiments, the pharmaceutical composition comprises 10 mM histidine. In some embodiments, the pharmaceutical composition comprises 3 mM methionine. In some embodiments, the pharmaceutical composition comprises 50 uM bisulfite salts. In some embodiments, the pharmaceutical composition comprises 10% sucrose (w/v). In some embodiments, the pharmaceutical composition comprises 0.01% polysorbate-20 (w/v). In some embodiments, the pharmaceutical composition further comprises 5% dextrose.

In some embodiments of the pharmaceutical compositions described herein, the anti-CD45 ADC is at a concentration of about 2 mg/mL. Accordingly, in a preferred embodiment, the pharmaceutical composition comprises an anti-CD45 ADC, 10 mM histidine, 3 mM methionine, 50 uM bisulfite salts, 10% (w/v) sucrose, and 0.01% (w/v) polysorbate-20 and has a pH of about 6.0.

The invention also includes a stable, pharmaceutical composition comprising an anti-CD45 ADC, histidine, sodium bisulfite, sucrose, methionine and polysorbate 20. In one embodiment, the pharmaceutical composition comprises about 25 to about 75 μM sodium bisulfite (e.g., 50 μM sodium bisulfite), about 5 to about 15 mM histidine (e.g., 10 mM histidine), about 5% to 10% sucrose (e.g., 10% sucrose), and about 1 to 8 mM methionine (e.g., 3 mM methionine), and polysorbate (e.g., 0.01% polysorbate 20), where the pharmaceutical composition has a pH of about 5.8 to about 6.2 (e.g., pH of 6).

As described in the examples, the pharmaceutical composition described herein is stable, where the anti-CD45 ADC is suitable for therapeutic use. Stability of the ADC can be assessed by techniques known in the art (including those disclosed in the Examples).

Stability, such as physical stability of a pharmaceutical disclosed herein (a formulation), may be assessed by methods well-known in the art, including measurement of a sample's apparent attenuation of light (absorbance, or optical density). Such a measurement of light attenuation relates to the turbidity of a formulation. The turbidity of a formulation is partially an intrinsic property of the protein dissolved in solution and is commonly determined by nephelometry, and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a formulation. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia, Fourth Ed., Directorate for the Quality of Medicine of the Council of Europe (EDQM), Strasbourg, France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability of a pharmaceutical protein are well-known in the art, e.g., size-exclusion chromatography or analytical ultracentrifucation.

A protein, e.g., an antibody, “retains its physical stability” in a pharmaceutical composition if it shows substantially no signs of instability, e.g., aggregation, precipitation and/or denaturation, upon visual examination of color and/or clarity or as measured by UV light scattering or by size exclusion chromatography. Aggregation is a process whereby individual protein molecules or complexes associate covalently or non-covalently to form aggregates. Aggregation can proceed to the extent that a visible precipitate is formed.

Examples of stability assays include measures of degradation such as aggregation. Aggregation is an important stability aspect for a pharmaceutical composition, as aggregates can cause immune reactions in patients receiving said composition. The pharmaceutical compositions disclosed herein provide for low levels of aggregate, making them suitable for therapeutic use and providing stability. “Aggregation” or “protein aggregation” in a pharmaceutical composition, as used herein, refers to aggregation of protein (antibodies which are included in the ADCs) in the formulation. The amount of protein in a formulation that are in an aggregated state, may be expressed, for example, as a percentage of the total protein content of the formulation. Aggregation may be detected, for example, by size exclusion chromatography (SEC) or other techniques that separate proteins in a solution based upon size or molecular weight.

In certain embodiments, the pharmaceutical composition disclosed herein has a low level of protein aggregate, e.g., less than 4% aggregate as determined by size exclusion chromatography (SEC). In one embodiment, the pharmaceutical composition disclosed herein comprises less than about 4% protein aggregate, less than about 3.5% protein aggregate, less than about 3% protein aggregate, less than about 2.5% protein aggregate, less than about 2% protein aggregate, or less than about 1.5% protein aggregate, as determined by SEC.

Aggregation can also be described in terms of the percentage of monomer in a solution, where a higher level or monomer indicates a low level of aggregate. Thus, in one embodiment, the pharmaceutical composition disclosed herein comprises more than about 90% monomer, more than about 91% monomer, more than about 92% monomer, more than about 93% monomer, more than about 94% monomer, more than about 95% monomer, more than about 96% monomer, more than about 97% monomer, more than about 98% monomer, and so forth, as determined by SEC.

Monomer and aggregation levels can also be tested using conditions to determine stability. The parameters described in the examples are included as assays that may be used to reflect the stability of a pharmaceutical composition disclosed herein, e.g., a pharmaceutical composition may have less than 40% aggregate, less than 30% aggregate, less than 20% aggregate following storage at 40 degrees Celsius for a given time period, e.g., 2 weeks, 3 weeks, or 4 weeks. Thus, aggregation can be used to define stability of a pharmaceutical composition where stability is defined as a level of protein aggregation in a protein formulation in reference to a storage assay, e.g., before a period of storage (e.g., the first protein formulation may be a formulation of protein of time zero (TO) when the formulation is initially prepared or of time zero before the formulation is subjected to a destabilizing condition, such as elevated temperature, and/or to long term storage.

In one aspect, disclosed herein are pharmaceutical compositions of an anti-CD45 ADC and a pharmaceutically acceptable carrier or the combination of excipients disclosed herein, wherein the ADC comprises an anti-CD45 antibody (Ab) conjugated to a sulfonated indolinobenzodiazepine pseudo dimer via a protease cleavable linker, wherein the sulfonated indolinobenzodiazepine pseudo dimer has a formula:

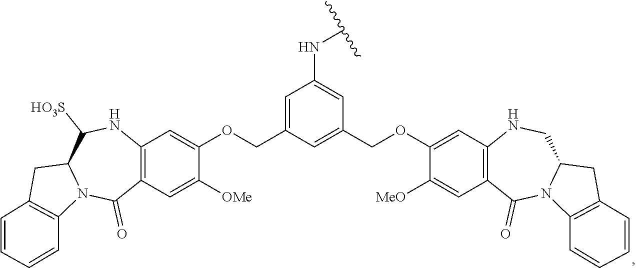

wherein the wavy line indicates the point of covalent attachment to the linker of the ADC, and wherein the anti-CD45 antibody comprises a heavy chain variable region comprising a CDR1 domain comprising the amino acid sequence as set forth in SEQ ID NO: 2, a CDR2 domain comprising the amino acid sequence as set forth in SEQ ID NO: 3, and a CDR3 domain comprising the amino acid sequence as set forth in SEQ ID NO: 4; and comprises a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO: 8. In some embodiments, the anti-CD45 antibody is an IgG1 isotype.

The pharmaceutical compositions described herein may be aqueous or lyophilized. In one embodiment, the pharmaceutical composition is lyophilized. The term “lyophilized” as used herein denotes a pharmaceutical composition which is freeze dried by any method known in the art. A lyophilized pharmaceutical composition can be reconstituted by dissolving said lyophilized pharmaceutical composition in a diluent such that the contents of the lyophilized powder are in solution, affording a “reconstituted pharmaceutical composition”. In some embodiments, the diluent is 5% dextrose.

In certain embodiments, a formulation is an aqueous formulation. The term “aqueous pharmaceutical composition” or “aqueous formulation” refers to a solution in which the solvent is water. In certain instances, an aqueous formulation refers to a liquid formulation in which the solvent is water wherein the formulation was not previously lyophilized, (i.e., does not result from reconstitution of a lyophilized formulation). In other circumstances, an aqueous formulation refers to a liquid formulation in which the solvent is water wherein the formulation was previously lyophilized (i.e., the formulation is a reconstituted formulation).

### Anti-CD45 Antibody-Drug Conjugate (ADC)

Anti-CD45 antibodies described herein can be conjugated (linked) to a cytotoxin via a linker, forming an antibody-drug conjugate (ADC).

### Anti-CD45 Antibody

In one aspect, the ADC comprises an anti-CD45 antibody, e.g., Ab5 antibody, is conjugated to a sulfonated indolinobenzodiazepine (IGN) pseudo dimer (e.g., DGN549) via a linker. In one embodiment, Ab5 is used as the anti-CD45 antibody. Antibody 5 (e.g., Ab5) binds to human CD45 (all isoforms) and can cross-react with CD45 from non-human primates (e.g., Cynomolgus CD45 and/or Rhesus CD45). Ab5 has diagnostic and therapeutic characteristics suitable for use in an ADC and the therapeutic uses described herein.

The amino acid sequences for the various binding regions of Ab5 are described in Table 10. Ab5 comprises a heavy chain variable region amino acid sequence as set forth in SEQ ID NO: 1. The heavy chain variable region CDR domains are set forth in SEQ ID NO: 2 (HC CDR1), SEQ ID NO: 3 (HC CDR2), and SEQ ID NO: 4 (HC CDR3). Ab5 comprises a light chain variable region amino acid sequence as set forth in SEQ ID NO: 5. The light chain variable region CDR domains are set forth in SEQ ID NO: 6 (LC CDR1), SEQ ID NO: 7 (LC CDR2), and SEQ ID NO: 8 (LC CDR3).

Accordingly, in some embodiments, the ADC used in the pharmaceutical composition described herein comprises an anti-CD45 antibody comprising a heavy chain variable region comprising a CDR1 domain comprising the amino acid sequence as set forth in SEQ ID NO: 2, a CDR2 domain comprising the amino acid sequence as set forth in SEQ ID NO: 3, and a CDR3 domain comprising the amino acid sequence as set forth in SEQ ID NO: 4; and comprising a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO: 8.

In some embodiments, the anti-CD45 antibody comprises a heavy chain variable region comprising the amino acid sequence as set forth in SEQ ID NO: 1, and a light chain variable region comprising the amino acid sequence as set forth in SEQ ID NO: 5. In some embodiments, the anti-CD45 antibody comprises a full length heavy chain amino acid sequence as set forth in SEQ ID NO: 9 and a full length light chain amino acid sequence as set forth in SEQ ID NO: 10. In some embodiments, the Ab5 antibody comprises a heavy chain full length DNA nucleotide sequence as set forth in SEQ ID NO: 11 and a light chain full length DNA nucleotide sequence as set forth in SEQ ID NO: 12.

An expression vector may contain a nucleic acid(s) encoding the heavy chain and/or light chain of an anti-CD45 antibody. For example, an expression vector may comprise the nucleotide sequence set forth in SEQ ID NO: 11 which encodes an anti-CD45 antibody heavy chain and/or the nucleotide sequence set forth in SEQ ID NO: 12 which encodes an anti-CD45 antibody light chain.

In some embodiments, the anti-CD45 antibody is an IgG1 isotype.

The Ab5 antibody described herein comprises mutations in the heavy chain constant region, including L234A, L235A, D265C, and H435A (EU Index). These mutations result in reduced Fc gamma receptor binding (e.g., Fc silencing mutations), provide a specific conjugation site for linker-cytotoxin, and/or reduce antibody half-life in vivo (e.g., enhance in vivo clearance). These Fc mutations with respect to antibody function are further described in WO 2020/086776, the entirety of which is incorporated by reference herein.

In one embodiment, the ADC described herein comprises an anti-CD45 antibody defined by the CDR, variable, or heavy and light chain amino acid sequences set forth in Table 10. In another embodiment, the ADC described herein comprises an anti-CD45 antibody defined by amino acid sequences as described in US Patent Publication No. US-2024-0254251, which is incorporated by reference herein.

In one embodiment, the anti-CD45 antibody is Ab1, as described in Table 10. Ab1 comprises a heavy chain variable region amino acid sequence as set forth in SEQ ID NO: 13. The heavy chain variable region CDR domains are set forth in SEQ ID NO: 14 (HC CDR1), SEQ ID NO: 15 (HC CDR2), and SEQ ID NO: 16 (HC CDR3). Ab1 comprises a light chain variable region amino acid sequence as set forth in SEQ ID NO: 17. The light chain variable region CDR domains are set forth in SEQ ID NO: 18 (LC CDR1), SEQ ID NO: 19 (LC CDR2), and SEQ ID NO: 20 (LC CDR3). The anti-CD45 antibody of the ADC described herein may include the foregoing sequences.

In one embodiment, the anti-CD45 antibody is Ab2, as described in Table 10. Ab2 comprises a heavy chain variable region amino acid sequence as set forth in SEQ ID NO: 23. The heavy chain variable region CDR domains are set forth in SEQ ID NO: 24 (HC CDR1), SEQ ID NO: 25 (HC CDR2), and SEQ ID NO: 26 (HC CDR3). Ab2 comprises a light chain variable region amino acid sequence as set forth in SEQ ID NO: 27. The light chain variable region CDR domains are set forth in SEQ ID NO: 28 (LC CDR1), SEQ ID NO: 29 (LC CDR2), and SEQ ID NO: 30 (LC CDR3). The anti-CD45 antibody of the ADC described herein may include the foregoing sequences.

In one embodiment, the anti-CD45 antibody is Ab3, as described in Table 10. Ab3 comprises a heavy chain variable region amino acid sequence as set forth in SEQ ID NO: 33. The heavy chain variable region CDR domains are set forth in SEQ ID NO: 34 (HC CDR1), SEQ ID NO: 35 (HC CDR2), and SEQ ID NO: 36 (HC CDR3). Ab3 comprises a light chain variable region amino acid sequence as set forth in SEQ ID NO: 37. The light chain variable region CDR domains are set forth in SEQ ID NO: 38 (LC CDR1), SEQ ID NO: 39 (LC CDR2), and SEQ ID NO: 40 (LC CDR3). The anti-CD45 antibody of the ADC described herein may include the foregoing sequences.

In one embodiment, the anti-CD45 antibody is Ab4, as described in Table 10. Ab4 comprises a heavy chain variable region amino acid sequence as set forth in SEQ ID NO: 43. The heavy chain variable region CDR domains are set forth in SEQ ID NO: 44 (HC CDR1), SEQ ID NO: 45 (HC CDR2), and SEQ ID NO: 46 (HC CDR3). Ab4 comprises a light chain variable region amino acid sequence as set forth in SEQ ID NO: 47. The light chain variable region CDR domains are set forth in SEQ ID NO: 48 (LC CDR1), SEQ ID NO: 49 (LC CDR2), and SEQ ID NO: 50 (LC CDR3). The anti-CD45 antibody of the ADC described herein may include the foregoing sequences.

In one embodiment, the anti-CD45 antibody is Ab6, as described in Table 10. Ab6 comprises a heavy chain variable region amino acid sequence as set forth in SEQ ID NO: 53. The heavy chain variable region CDR domains are set forth in SEQ ID NO: 54 (HC CDR1), SEQ ID NO: 55 (HC CDR2), and SEQ ID NO: 56 (HC CDR3). Ab6 comprises a light chain variable region amino acid sequence as set forth in SEQ ID NO: 57. The light chain variable region CDR domains are set forth in SEQ ID NO: 58 (LC CDR1), SEQ ID NO: 59 (LC CDR2), and SEQ ID NO: 60 (LC CDR3). The anti-CD45 antibody of the ADC described herein may include the foregoing sequences.

In one embodiment, the anti-CD45 antibody is Ab7, as described in Table 10. Ab7 comprises a heavy chain variable region amino acid sequence as set forth in SEQ ID NO: 63. The heavy chain variable region CDR domains are set forth in SEQ ID NO: 64 (HC CDR1), SEQ ID NO: 65 (HC CDR2), and SEQ ID NO: 66 (HC CDR3). Ab7 comprises a light chain variable region amino acid sequence as set forth in SEQ ID NO: 67. The light chain variable region CDR domains are set forth in SEQ ID NO: 68 (LC CDR1), SEQ ID NO: 69 (LC CDR2), and SEQ ID NO: 70 (LC CDR3). The anti-CD45 antibody of the ADC described herein may include the foregoing sequences.

### Indolinobenzodiazepine (IGN)

Indolinobenzodiazepine dimers represent a chemical class of cytotoxins with high in vitro potency (low pM range IC₅₀ values) towards cancer cells. Similar to the PBD dimer SJG-136, IGN dimers bind to the minor groove of DNA, and covalently bind to guanine residues via the two imine functionalities in the dimer, resulting in crosslinking of the DNA. An IGN dimer (IGN 6; replacing the methylene groups of the PBD moiety with phenyl rings) demonstrated ~10-fold higher potency in vitro as compared to SJG-136, possibly due to faster rate of adduct formation with DNA IGN (see, e.g., Miller et al., “A New Class of Antibody-Drug Conjugates with Potent DNA Alkylating Activity” Mol. Cancer Ther. 2016, 15(8), 1870-1878). In contrast, IGN pseudo dimers comprise a single reactive indolinobenzodiazepine imine; the second indolinobenzodiazepine in the dimeric cytotoxin is present in reduced (amine) form. Accordingly, IGN pseudo dimers alkylate DNA through the single imine moiety present in the dimer, and do not crosslink DNA.

In some embodiments, the cytotoxin used in the ADC described herein is an IGN pseudo dimer having a structure of formula:

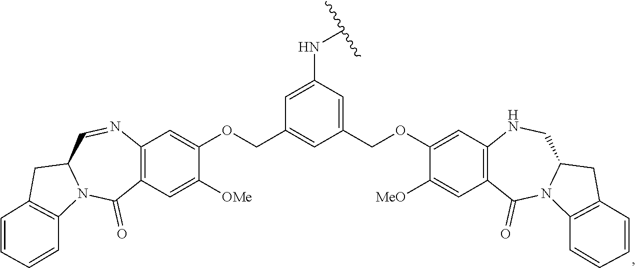 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker. The foregoing structure is also referenced as DGN549.

In some embodiments, the cytotoxin used in an ADC described herein is a sulfonated IGN pseudo dimer having a structure of formula:

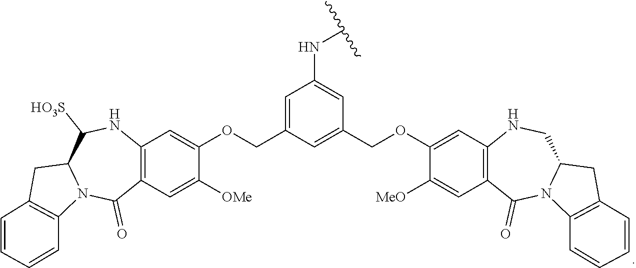

wherein the wavy line indicates the attachment point of the linker. The foregoing structure is also referenced as sulfonated DGN549.

In some embodiments, the cytotoxin-linker conjugate, prior to conjugation to the antibody and including the reactive substituent Z′, taken together as Cy-L-Z′, has the structure:

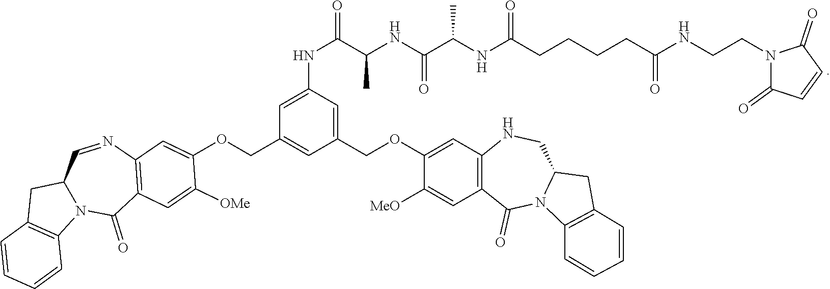 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

The foregoing cytotoxin-linker conjugate is referred to herein as DGN549-C and is used for cysteine conjugation.

DGN549 is present in the ADC IMGN632, which is disclosed in, for example, International Patent Application Publication No. WO2017004026, which is incorporated by reference herein. Conjugation of DGN549-C to an antibody is described in Bai et al. (2020) Bioconjugate Chem 31: 93-103.

In some embodiments, the cytotoxin is a sulfonated indolinobenzodiazepine pseudo dimer having a structure of formula:

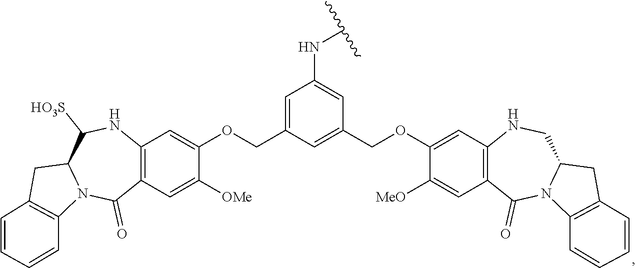

wherein the wavy line indicates the point of attachment to the linker. This cytotoxin is a sulfonated version of the DGN549 cytotoxin reversibly sulfonated at the imine moiety. DGN549 is present in the ADC IMGN632, which is disclosed in, for example, International Patent Application No. WO 2017/004023, which is incorporated by reference herein.

Sulfonation of DGN549 can be achieved by exposure to high concentrations of bisulfite salts. This reversible sulfonation, which is maintained in the presence of excess bisulfite salts, enhances the water solubility of the molecule (Miller et al. (2016) Mo Cancer Ther 15(8): 1870-1878). The sulfonation of the imine moiety is reversible in the absence of excess concentrations of the bisulfite salts. Miller et al. (2016) Mo Cancer Ther 15(8): 1870-1878 reported that a sulfonated IGN (corresponding to a sulfonated DGN549 described herein) spontaneously reversed to the free imine ex vivo in human plasma.

For an anti-CD45 ADC, in one embodiment, a sulfonated indolinobenzodiazepine pseudo dimer is attached to a linker to form a cytotoxin-linker conjugate. Conjugation of DGN549 to an antibody is described in Bai et al., (2020) Bioconjugate Chem 31: 93-103.

In some embodiments, prior to conjugation to the antibody, the sulfonated DGN549 cytotoxin-linker conjugate has the structure:

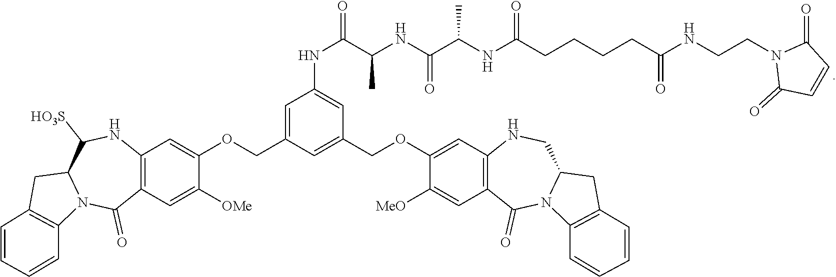

In one embodiment, an anti-CD45 ADC comprising the sulfonated indolinobenzodiazepine pseudo dimer for use in the pharmaceutical compositions disclosed herein has the formula:

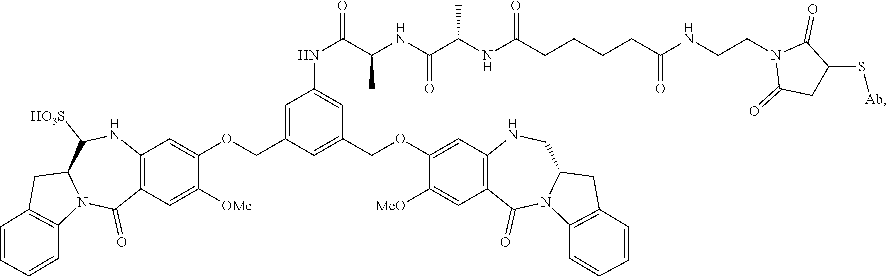

wherein Ab is the anti-CD45 antibody.

In one embodiment, an IGN pseudo dimer described herein (e.g., DGN549) is conjugated to an anti-CD45 antibody such that the ratio of toxin:antibody is about 1.8 to 2.2 In certain embodiments, the average number of toxins attached to the anti-CD45 antibody is 1.8 to 2.0. In certain embodiments, the average number of toxins attached to the anti-CD45 antibody is 1.7 to 2.1. In certain embodiments, the average number of toxins attached to the anti-CD45 antibody is 1.6 to 2.2. In one embodiment, an IgG1 anti-CD45 antibody is conjugated to two IGN pseudo dimers (e.g., DGN549), wherein the anti-CD45 antibody comprises a heavy chain variable region comprising a CDR1 domain comprising the amino acid sequence as set forth in SEQ ID NO:2, a CDR2 domain comprising the amino acid sequence as set forth in SEQ ID NO:3, and a CDR3 domain comprising the amino acid sequence as set forth in SEQ ID NO:4; and comprises a light chain variable region comprising a CDR1 domain comprising the amino acid sequence as set forth in SEQ ID NO:6, a CDR2 domain comprising the amino acid sequence as set forth in SEQ ID NO: 7; and a CDR3 domain comprising the amino acid sequence as set forth in SEQ ID NO:8. In a preferred embodiment, the toxin is conjugated to an anti-CD45 antibody via a cysteine residue in the Fc region of the antibody and via a linker, such as a protease-cleavable linker.

The term “linker” as used herein means a divalent chemical moiety comprising a covalent bond or a chain of atoms that covalently attaches an anti-CD45 antibody to a cytotoxin (e.g., sulfonated DGN549) to form an anti-CD45 ADC. Suitable linkers have two reactive termini, one for conjugation to an antibody and the other for conjugation to a cytotoxin.

In one aspect, the linker is susceptible to enzymatic hydrolysis, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. In a preferred embodiment, the peptide linker comprises an alanine dipeptide (Ala-Ala).

The anti-CD45 ADCs disclosed herein comprise a protease-cleavable linker. In one aspect, the linker has a formula:

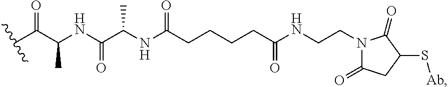

wherein the wavy line indicates the point of attachment to a sulfonated indolinobenzodiazepine pseudo dimer and Ab is anti-CD45 antibody Ab5. In some embodiments, the linker is conjugated to the antibody at a cysteine in the Fc region of the antibody. In a preferred embodiment, the cysteine is at position 265 (numbering according to the EU index according to Kabat).

In some embodiments, the linker, prior to covalent attachment to the antibody, comprises as a reactive substituent a maleimide (reacting with a cysteine sulfur atom of the antibody to form an S-substituted succinimide). In some embodiments, the linker comprising a maleimide, prior to covalent attachment to the antibody with the reactive substituent, has a formula:

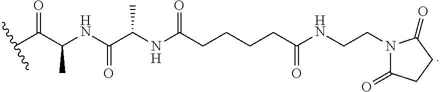 [SMILES: *C(=O)[C@H](C)NC(=O)[C@H](C)NC(=O)CCCCC(=O)NCCN1C(=O)CCC1=O]

wherein the wavy line indicates the point of covalent attachment to a sulfonated indolinobenzodiazepine pseudo dimer. Maleimide-thiol chemistry and its uses in conjugation are well described in the art (see e.g., Ravasco et al., (2019), Chemistry 25(1):43-59).

In a one embodiment, the pharmaceutical compositions disclosed herein comprise an ADC having the following formulae (DGN549-C), where Ab is the anti-CD45 antibody (e.g., Ab5) conjugated to the linker-toxin via a cysteine residue:

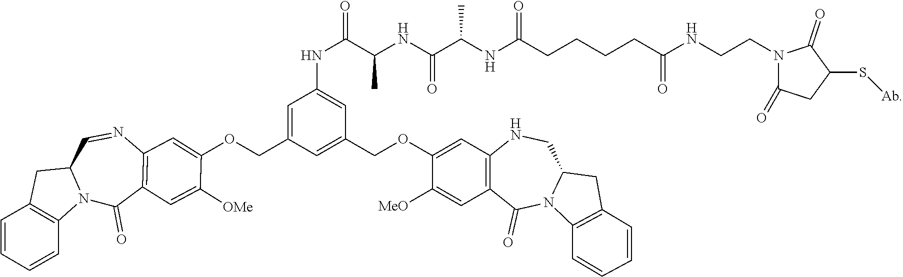

In a one embodiment, the pharmaceutical compositions disclosed herein and ADC having the following formulae (sulfonated DGN549-C), where Ab is the anti-CD45 antibody (e.g., Ab5) conjugated to the linker-toxin via a cysteine residue:

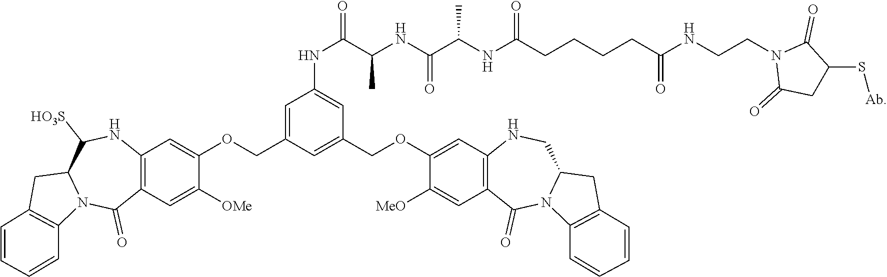

In a one embodiment, the pharmaceutical compositions disclosed herein comprise a mixture of the following formulae (DGN549-C and sulfonated DGN549-C), where Ab is an anti-CD45 antibody (e.g., Ab5) conjugated to the linker-toxin via a cysteine residue:

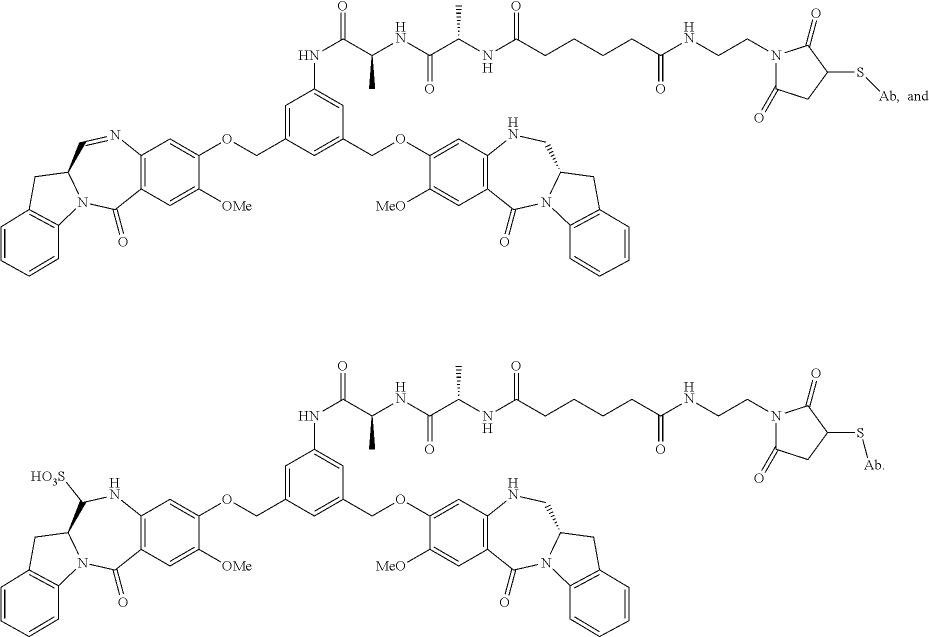

Examples of benzodiazepine toxins that may be used herein (as well as method of making the same) include those set forth in U.S. Pat. Nos. 9,669,102; 9,974,867; 10,208,127; 9,555,125; 9,617,270; and 8,765,740, each which is incorporated by reference herein. Additional benzodiazepine toxins, as well as methods for making benzodiazepine toxins, are described in U.S. Pat. Nos. 9,873,708; 11,390,633; 11,274,121; 10,081,640; and 9,534,000, each of which is incorporated by reference herein. Cysteine conjugated conjugates and methods conjugating are described in U.S. Pat. No. 10,898,579, which is incorporated by reference herein. Sulfonated benzodiazepines and related methods that may be used herein are described in U.S. Pat. No. 10,287,256, which is incorporated by reference herein. Anti-CD45 ADCs comprising IGNs and uses thereof, are described in WO 2024/064771, which is incorporated by reference herein.

### Linkers

A variety of linkers can be used to conjugate the anti-CD45 antibodies described to a cytotoxic molecule. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a dipeptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, or antibody fragments, described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

One of skill in the art will recognize that a reactive substituent Z′ attached to the linker and a reactive substituent on the antibody are engaged in the covalent coupling reaction to produce the chemical moiety Z, and will recognize the reactive moiety Z′. Therefore, ADCs useful in conjunction with the methods described herein may be formed by the reaction of an antibody with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

Examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substituents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive moiety Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others.

For instance, linkers suitable for the synthesis of ADCs include, without limitation, reactive substituents Z′ such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

The foregoing linker moieties, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

### Preparation of Antibody-Drug Conjugates

In the ADCs as disclosed herein, an anti-CD45 antibody, or antigen binding fragment thereof, is conjugated to one or more cytotoxins, e.g. about 1 to about 20 cytotoxins per antibody, through a linker L as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z′, followed by reaction with a reactive substituent of an antibody or antigen binding fragment thereof as described herein above. Additional methods for preparing ADC are described herein.

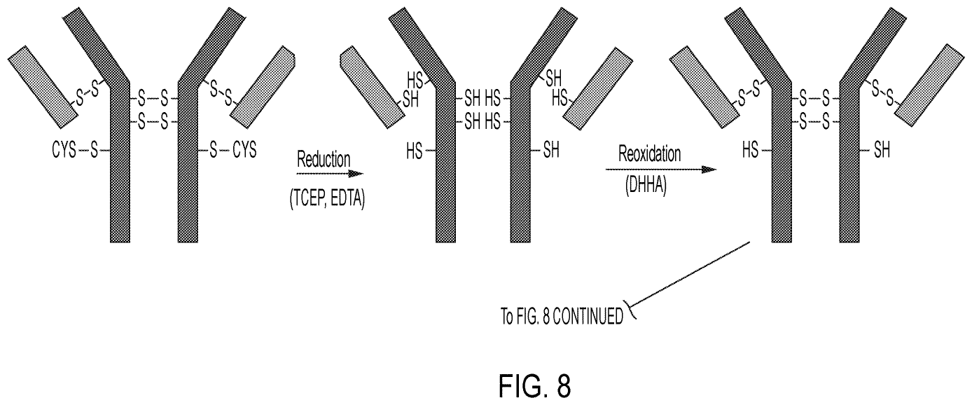

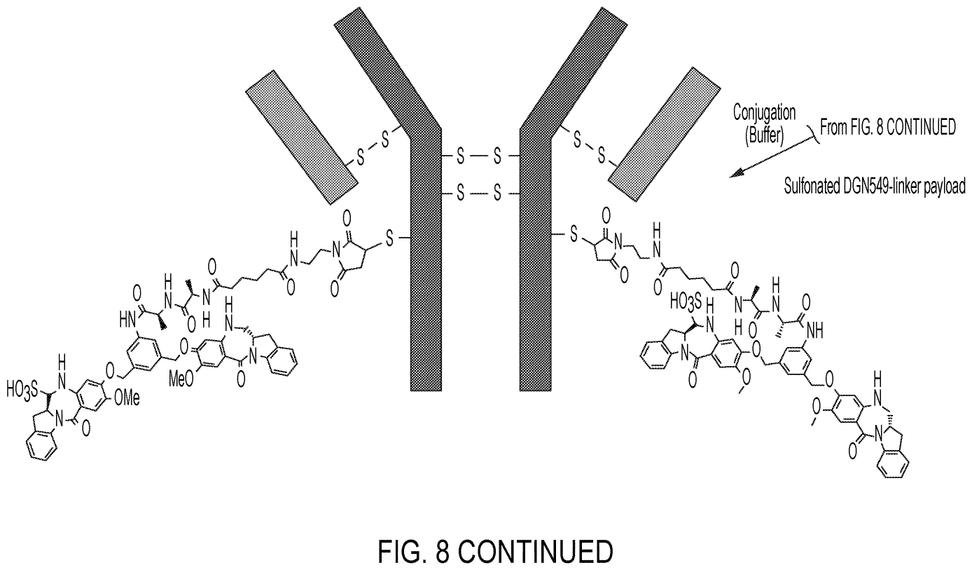

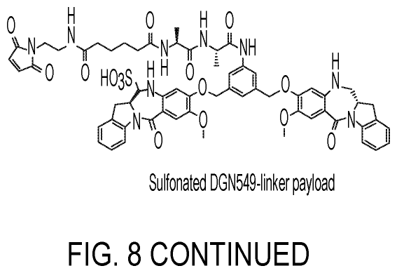

In one embodiment, an ADC of the disclosure comprising an anti-CD45 antibody and a DGN549 cytotoxin is produced through the conjugation of an anti-CD45 antibody to a DGN549 linker-payload via a succinimide linkage. In this embodiment, conjugation is performed via reaction of a maleimide at the linker terminus with an engineered cysteine residue (or pair of engineered cysteine residues) in the antibody. A schematic illustration according to a non-limiting embodiment is provided in FIG. 8. With reference to FIG. 8, the method generally comprises reduction of the anti-CD45 antibody with e.g., tris(2-carboxyethyl)phosphine (TCEP) followed by selective reoxidation of the interchain cysteines to re-form the native IgG1 disulfide bonds using, e.g., dehydroascorbic acid (DHAA). The engineered cysteines (at amino acid position 265 of the Fc region (EU numbering per Kabat) remain reduced and are then conjugated with the DGN549-linker payload conjugate (DGN549-C, which is DGN549 with a protease cleavable linker). In some embodiments, and as illustrated in FIG. 8, in order to enhance solubility of the cytotoxin linker conjugate, and ADC, the DGN549 cytotoxin is reversibly sulfonated by exposure to excess sodium bisulfite and is maintained in the sulfonated state by including excess bisulfite in a buffered conjugation medium. This general methodology is disclosed in, for example, U.S. Pat. No. 10,287,256 to Hilderbrand et al., and is incorporated by reference herein with respect to preparation of ADC's.

In another aspect, the anti-CD45 antibody, or antigen binding fragment thereof, has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-CD45 antibody, or antigen binding fragment thereof, can have one or more carbohydrate groups that can be chemically modified to have one or more sulfhydryl groups. The ADC is then formed by conjugation through the sulfhydryl group's sulfur atom as described herein above.

In yet another aspect, the anti-CD45 antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to anti-CD45 antibodies are found, for example, in U.S. Pat. Nos. 5,208,020; 6,441,163; WO2005037992; WO2005081711; and WO2006/034488, all of which are hereby expressly incorporated by reference in their entirety.

### Therapeutic Uses

CD45 is an important cell surface molecule broadly expressed throughout the hematopoietic and immune systems. Anti-CD45 ADCs (particularly an ADC comprising an anti-CD45 antibody conjugated via a protease cleavable linker to an IGN, e.g. DGN549), described herein are used as conditioning agents in human patients who are in need of a stem cell transplant, e.g., a hematopoietic stem cell (HSC) transplant. There is currently a need for compositions and methods for promoting the engraftment of stem cell transplants, e.g., exogenous hematopoietic stem cell grafts such that the multi-potency and hematopoietic functionality of these cells is preserved following transplantation. The pharmaceutical compositions disclosed herein provide a solution to this challenging problem.

Described herein are pharmaceutical compositions of anti-CD45 ADCs that can be used to treat patients with conditions for which depletion of CD45+ cells is beneficial, including, but not limited to, leukemias and lymphomas, as well as patients hemoglobinopathy disorders, including sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S-C disease, Fanconi anemia, aplastic anemia, or Wiskott-Aldrich syndrome.

By targeting CD45 expressing cells with anti-CD45 ADCs described herein, generally both hematopoietic stem cells (HSCs) and leukocytes can be depleted (CD45 is a pan leukocyte marker). Thus, in certain embodiments, provided herein is a method for providing an immune reset in a subject in need thereof. For example, by administering an anti-CD45 ADC described herein to a patient having a disease associated with disease causing leukocytes, e.g., an autoimmune disease, the disease causing leukocytes can be eliminated (along with the HSCs) and the patient can then build a new immune system from subsequently transplanted HSCs.

An additional benefit of the CD45 specific ADCs described herein is that, as opposed to the non-targeted highly toxic chemotherapies commonly used in conditioning and treatment, red blood cells should be unaffected in the patient given that red blood cells do not generally express CD45.

Thus, disclosed herein are methods of treating a variety of disorders, such as diseases of a cell type in the hematopoietic lineage, cancers, autoimmune diseases, metabolic disorders, and stem cell disorders, among others. In certain embodiments, an anti-CD45 ADC described herein is used as a conditioning agent so as to deplete the CD45+ cells in the human subject prior to a stem cell transplant for treatment.

Thus, the compositions and methods described herein may (i) directly deplete a population of CD45 expressing cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of an anti-CD45 ADC capable of binding an endogenous disease-causing cell that expressed CD45 or a hematopoietic stem cell. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. In certain embodiments, ADCs, capable of binding CD45 can be administered to a patient to effect both of the foregoing activities. ADCs, that bind CD45 antigen expressed by immune cells, e.g., hematopoietic stem cells, can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

In one embodiment, a pharmaceutical composition of an anti-CD45 ADC described herein is administered to a human patient for conditioning wherein the patient has acute myelogenous leukemia. In one embodiment, a pharmaceutical composition of an anti-CD45 ADC described herein is administered to a human patient for conditioning wherein the patient has myelodysplastic syndrome. In certain embodiments, a pharmaceutical composition of an anti-CD45 ADC described herein is administered to a human patient having poor prognosis acute myeloid leukemia (AML), high risk myelodysplastic syndrome (MDS) or myeloproliferative neoplasm (MPN).

Following conditioning with an anti-CD45 ADC (i.e., following administration of a pharmaceutical composition of an anti-CD45 ADC described herein), hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The pharmaceutical compositions described herein can thus be used to treat a non-malignant hemoglobinopathy (e.g., a hemoglobinopathy selected from the group consisting of sickle cell anemia, thalassemia (e.g., alpha thalassemia, beta thalassemia, non-transfusion dependent beta thalassemia (NTDT), thalassemia intermedia, thalassemia major), hemoglobin C disease, hemoglobin S-C disease, Fanconi anemia, aplastic anemia, and Wiskott-Aldrich syndrome).

Transplanted cells can be autologous or allogeneic. HSC transplants can be from bone marrow, peripheral blood, or cord blood.

Additionally or alternatively, the pharmaceutical compositions described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma. In some embodiments, the hematological cancer is a relapsed and/or refractory acute myeloid leukemia (AML). In some embodiments, the hematological cancer is a T cell lymphoma (e.g., a recurrent T cell lymphoma).

Additionally or alternatively, the pharmaceutical compositions described herein can be used to administer genetically-modified stem cells to a patient suffering from a condition that results from a defective gene (e.g., a mutation). For instance, the genome of living cells (e.g., stem cells) can be modified for therapeutic purposes. In particular, a therapeutic effect can be achieved by correcting a defective gene prior to engraftment of the genetically-modified stem cells back into the patient. In some embodiments, HSCs may be extracted from a patient suffering from a disorder caused by a defective gene and purified using methods known in the art. The isolated cells can be treated ex vivo using known methods in the art and its genome can be modified as desired e.g., edited to correct the defective target gene into a functional gene. The genetically-modified stem cells are subsequently administered back to the patient who has been conditioned using the compositions and methods described herein. In some embodiments, the stem cells may be allogeneic to the patient to whom they are administered.

Additionally or alternatively, the pharmaceutical compositions described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additional diseases that can be treated with the pharmaceutical compositions described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The pharmaceutical compositions disclosed herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the pharmaceutical compositions described herein may be used to induce transplant tolerance in a solid organ transplant recipient (e.g., a kidney transplant, lung transplant, liver transplant, and heart transplant, among others). The pharmaceutical compositions described herein are well-suited for use in the induction of solid organ transplant tolerance, for instance, because a low percentage temporary or stable donor engraftment is sufficient to induce long-term tolerance of the transplanted organ.

In some embodiments, the transplant is allogeneic. In some embodiments, the transplant is autologous.

In some embodiments, the transplant is a bone marrow transplant, a peripheral blood transplant, or a cord blood transplant.

In some embodiments, the transplant includes hematopoietic cells (e.g., hematopoietic stem cells).

In some embodiments, the transplant includes gene-edited stem cells.

In any of the embodiments described herein, the transplant may be any solid organ or skin transplant. In some embodiments, the transplant is selected from the group consisting of kidney transplant, heart transplant, liver transplant, pancreas transplant, lung transplant, intestine transplant, and skin transplant.

In addition, the pharmaceutical compositions described herein can be used to treat cancers directly, such as cancers characterized by cells that are CD45+. For instance, the pharmaceutical compositions described herein can be used to treat leukemia, such as in patients that exhibit CD45+ leukemic cells. By depleting CD45+ cancerous cells, such as leukemic cells, the pharmaceutical compositions described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma. In some embodiments, the cancer is a relapsed and/or refractory acute myeloid leukemia (AML). In some embodiments, the cancer is a recurrent T cell lymphoma.

In addition, the pharmaceutical compositions described herein can be used to treat autoimmune disorders. For instance, the pharmaceutical compositions disclosed herein can be administered to a subject, such as a human patient suffering from an autoimmune disorder, so as to kill a CD45+ immune cell. For example, a CD45+ immune cell may be an autoreactive lymphocyte, such as a T-cell that expresses a T-cell receptor that specifically binds, and mounts an immune response against, a self-antigen. By depleting self-reactive, CD45+ cells, the pharmaceutical compositions described herein can be used to treat autoimmune diseases, such as those described below. Additionally or alternatively, the pharmaceutical compositions described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the pharmaceutical compositions disclosed herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjogren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

The pharmaceutical compositions disclosed herein may be administered by a variety of routes, such as subcutaneously, intravenously, or parenterally. In some embodiments, the pharmaceutical composition is administered intravenously to a human subject.

The pharmaceutical composition comprising an anti-CD45 ADC may be administered one or more times (e.g., 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, the antibody, or antigen-binding fragment thereof can be administered to the patient at a time that optimally promotes engraftment of the exogenous hematopoietic stem cells, for instance, from 1 hour to 1 week (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

In one embodiment, the pharmaceutical composition is administered about 10 days prior to the cell transplant. In one embodiment, an HSC transplant is administered to a patient in need thereof 14 days following administration of a pharmaceutical composition disclosed herein. Alternatively, an HSC transplant is administered 14 days after a single dose administration of a pharmaceutical composition described herein, but no later than 28 days following administration of the pharmaceutical composition. Thus, in certain embodiments, there are 14 to 28 days between administration of the pharmaceutical composition and delivery of the HSC transplant.

The pharmaceutical compositions described herein can be administered in an amount sufficient to reduce the quantity of the target CD45 expressing cells. For example, the pharmaceutical compositions described herein can be administered in an amount sufficient to reduce the quantity of endogenous CD45+ cells in the bone marrow and/or in the peripheral blood by, for example, about 10%, 20%, 30%, 40%, 50%, 60%, 70%, 80%, 90%, 95%, or more. In some embodiments, the anti-CD45 antibody, antigen-binding fragment thereof, or ADC can be administered in an amount sufficient to reduce the quantity of endogenous hematopoietic stem cells, for example, by 10%, 20%, 30%, 40%, 50%, 60%, 70%, 80%, 90%, 95%, or more prior to hematopoietic stem cell transplant therapy. The reduction in hematopoietic stem cell count can be monitored using conventional techniques known in the art, such as by FACS analysis of cells expressing characteristic hematopoietic stem cell surface antigens in a blood sample withdrawn from the patient at varying intervals during conditioning therapy. For instance, a physician of skill in the art can withdraw a blood sample from the patient at various time points during conditioning therapy and determine the extent of endogenous hematopoietic stem cell reduction by conducting a FACS analysis to elucidate the relative concentrations of hematopoietic stem cells in the sample using antibodies that bind to hematopoietic stem cell marker antigens. According to some embodiments, when the concentration of the HSCs has reached a minimum value in response to the conditioning therapy with a pharmaceutical composition disclosed herein, the physician may conclude the conditioning therapy and begin preparing the patient for HSC transplant therapy.

In certain embodiments, the pharmaceutical compositions disclosed herein can be administered to the patient at a time that optimally promotes engraftment of the exogenous hematopoietic stem cells, for instance, from about 1 hour to about 1 week (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

The pharmaceutical compositions described herein may be used as a monotherapy conditioning agent. A monotherapy conditioning agent is an agent used in the absence of other myeloablative agents, e.g., busulfan. In one embodiment, the pharmaceutical compositions described herein are used as a monotherapy conditioning agent prior to allogeneic or autologous hematopoietic stem cell (HSC) transplantation.

Following conclusion of conditioning therapy, the patient may then receive an infusion (e.g., an intravenous infusion) of exogenous hematopoietic stem cells, such as from the same physician that performed the conditioning therapy or from a different physician. The physician may administer the patient an infusion of autologous, syngeneic, or allogeneic hematopoietic stem cells, for instance, at a dosage of from about 1×10³ to about 1×10⁹ hematopoietic stem cells/kg. The physician may monitor the engraftment of the hematopoietic stem cell transplant, for example, by withdrawing a blood sample from the patient and determining the increase in concentration of hematopoietic stem cells or cells of the hematopoietic lineage (such as megakaryocytes, thrombocytes, platelets, erythrocytes, mast cells, myeloblasts, basophils, neutrophils, eosinophils, microglia, granulocytes, monocytes, osteoclasts, antigen-presenting cells, macrophages, dendritic cells, natural killer cells, T-lymphocytes, and B-lymphocytes) following administration of the transplant. This analysis may be conducted, for example, from about 1 hour to about 6 months, or more, following hematopoietic stem cell transplant therapy (e.g., about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, or more). A finding that the concentration of hematopoietic stem cells or cells of the hematopoietic lineage has increased (e.g., by about 1%, 2%, 3%, 4%, 5%, 6%, 7%, 8%, 9%, 10%, 20%, 30%, 40%, 50%, 60%, 70%, 80%, 90%, 100%, 200%, 500%, or more) following the transplant therapy relative to the concentration of the corresponding cell type prior to transplant therapy provides one indication that treatment with the anti-CD45 ADC has successfully promoted engraftment of the transplanted hematopoietic stem cell graft. The foregoing may also be used in therapies relating to CD45 cell; depletion, e.g., HSC and immune cell depletion for treatment of an autoimmune disease, or for treatment of a hematological cancer.

Engraftment of hematopoietic stem cell transplants due to the administration of a pharmaceutical composition disclosed herein can manifest in a variety of empirical measurements. For instance, engraftment of transplanted hematopoietic stem cells can be evaluated by assessing the quantity of competitive repopulating units (CRU) present within the bone marrow of a patient following administration of the pharmaceutical composition and subsequent administration of an HSC transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample. In some embodiments, the conditioning methods described herein are effective to achieve polyclonal engraftment.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

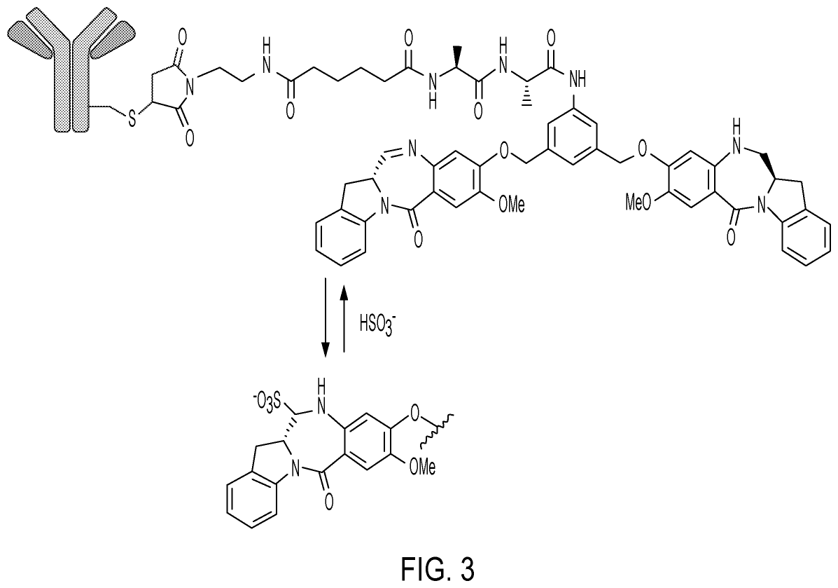

Formulation studies in the examples below were performed using an anti-CD45 ADC comprising anti-CD45 Ab5 conjugated to DGN-549 via a cysteine in the heavy chain of the antibody at amino acid 265 (EU numbering by Kabat) in the Fc region of the antibody. DGN-549 is described in FIG. 3. The antibody sequences of an exemplary CD45 antibody used in the ADC described in the examples are provided in Table 10 (the exemplary antibody is Ab5).

### Example 1: Anti-CD45 ADC Formulation Testing to Determine Buffer

A number of formulations were tested to determine which formulation was able to stabilize anti-CD45 ADC. Four formulations were tested as outlined in Table 1:

TABLE 1
Anti-CD45 ADC Formulations

| Formulation No. | Excipients and pH | ADC Concentration |
|---|---|---|
| F1a | 10 mM Histidine | 0.5 mg/mL |
| (F1 0.5 mg/ml) | pH 6 | |
| | 10% Sucrose | |
| | 50 μM Sodium Bisulfite | |
| | 3 mM Methionine | |
| | 0.01% polysorbate-20 | |
| F1b | 10 mM Histidine | 2 mg/mL |
| (F1 2 mg/ml) | pH 6 | |
| | 10% Sucrose | |
| | 50 μM Sodium Bisulfite | |
| | 3 mM Methionine | |
| | 0.01% polysorbate-20 | |
| F2 | 10 mM Succinate | 2 mg/mL |
| | pH 4.2 | |
| | 7.2% Trehalose | |
| | 50 μM Sodium Bisulfite | |
| | 3 mM Methionine | |
| | 0.01% polysorbate-20 | |
| F3 | 10 mM Acetate | 2 mg/mL |
| | pH 4.2 | |
| | 10% Sucrose | |
| | 50 μM Sodium Bisulfite | |
| | 3 mM Methionine | |
| | 0.01% polysorbate-20 | |

Formulations F1a and F1b were the same except F1a had an ADC concentration of 0.5 mg/ml and F1b had an ADC concentration of 2 mg/ml. The formulations in Table 1 varied by buffer, pH and in some cases sugar. Formulations F1a and F1b each contained a histidine buffer, and each formulation had a pH of 6. The F2 Formulation included the methionine, sodium bisulfite and polysorbate 20 (same excipients tested in Formulations F1a and F1b) but included a succinate buffer at a pH of 4.2 Formulation F2 also included trehalose instead of sucrose, as tested in Formulations F1a, Fib, and F3. Formulation F3 included acetate buffer.

Stability of each of the formulations was evaluated for (i) visual appearance, (ii) concentration, (iii) turbidity, (iii) size-exclusion chromatography (SEC) (which tests aggregation), and (iv) capillary electrophoresis sodium dodecyl sulfate (CE-SDS) under non-reducing conditions. Formulations were tested under five different temperatures, including −80° C., −20° C., 2-8° C., 25° C., and 40° C. Formulations at each temperature were evaluated under the above criteria after 2 weeks at the respective temperatures and after 1 month.

The formulations were also tested under freeze/thaw (F/T) conditions according to 1, 3 and 5 cycles of F/T at temperatures of −80° C. and 25° C.

Results from the testing are provided below:

### Visual Appearance

No visible particles were observed in any of the samples across all conditions tested.

### Concentration

The concentration results generated by a high throughput Lunatic instrument (at A280 and A330) are described in Table 2. No trending was observed among the different formulations. Multiple F/T cycles and stress conditions were determined to have no effect on protein (ADO) concentration.

TABLE 2
Anti-CD45 ADC Concentration

| | | | Concentration (mg/mL) | | | |
|---|---|---|---|---|---|---|
| Condition | Time Point | | F1a | F1b | F2 | F3 |
| T = 0 | | | 2.14 | 0.47 | 2.24 | 2.16 |
| Freeze/Thaw | 1X FT | | 1.86 | 0.51 | 1.85 | 1.90 |
| (FT) | 3X FT | | 1.93 | 0.44 | 1.73 | 1.85 |
| | 5X FT | | 1.83 | 0.45 | 1.93 | 1.90 |
| 2 Weeks | −80 | ° C. | 1.88 | 0.42 | 1.91 | 1.91 |
| Storage | −20 | ° C. | 1.86 | 0.42 | 1.95 | 1.91 |
| | 2 to 8 | ° C. | 1.89 | 0.39 | 1.95 | 1.89 |
| | 25 | ° C. | 1.83 | 0.44 | 1.96 | 1.92 |
| | 40 | ° C. | 1.88 | 0.43 | 2.03 | 1.99 |
| 1 Month | −80 | ° C. | 2.00 | 0.48 | 2.23 | 2.15 |
| Storage | −20 | ° C. | 2.04 | 0.49 | 2.21 | 2.04 |
| | 2 to 8 | ° C. | 2.09 | 0.51 | 1.93 | 2.09 |
| | 25 | ° C. | 2.05 | 0.49 | 2.31 | 2.18 |
| | 40 | ° C. | 2.08 | 0.50 | 2.39 | 2.00 |

### Turbidity

The turbidity of the formulation was measured using a UV-Vis Spectrophotometer at 420 nm following exposure to the freeze/thaw and temperature conditions. Results are provided in Table 3.

TABLE 3
Anti-CD45 ADC Turbidity

| | | | Turbidity (A420) | | | |
|---|---|---|---|---|---|---|
| Condition | Time Point | | F1a | F1b | F2 | F3 |
| T = 0 | | | 0.03 | 0.00 | 0.04 | 0.03 |
| Freeze/Thaw | 1X FT | | 0.01 | 0.02 | 0.01 | 0.04 |
| (FT) | 3X FT | | 0.08 | 0.02 | 0.00 | 0.03 |
| | 5X FT | | 0.05 | 0.00 | 0.00 | 0.03 |
| 2 Weeks | −80 | ° C. | 0.03 | 0.00 | 0.05 | 0.03 |
| Storage | −20 | ° C. | 0.04 | 0.00 | 0.04 | 0.03 |
| | 2 to 8 | ° C. | 0.02 | 0.04 | 0.03 | 0.02 |
| | 25 | ° C. | 0.09 | 0.02 | 0.03 | 0.04 |
| | 40 | ° C. | 0.05 | 0.01 | 0.06 | 0.05 |
| 1 Month | −80 | ° C. | 0.06 | 0.03 | 0.02 | 0.04 |
| Storage | −20 | ° C. | 0.08 | 0.03 | 0.03 | 0.02 |
| | 2 to 8 | ° C. | 0.05 | 0.03 | 0.09 | 0.03 |
| | 25 | ° C. | 0.18 | 0.05 | 0.04 | 0.03 |
| | 40 | ° C. | 0.16 | 0.04 | 0.11 | 0.10 |

AQ

A minor increase in turbidity with Fla incubated at 40° C. and 25° C. for 1 month was observed without an impact on formulation quality. A minor increase in turbidity with F2 and F3 at 40° C. for 1 month was observed but had no impact on formulation quality.

### Size Exclusion Chromatography (SEC)

The percent monomer (% Monomer), percent high molecular weight species (% HMW), and percent low molecular weight species (% LMW) were measured using SEC following exposure to either the freeze/thaw or the various temperature conditions.

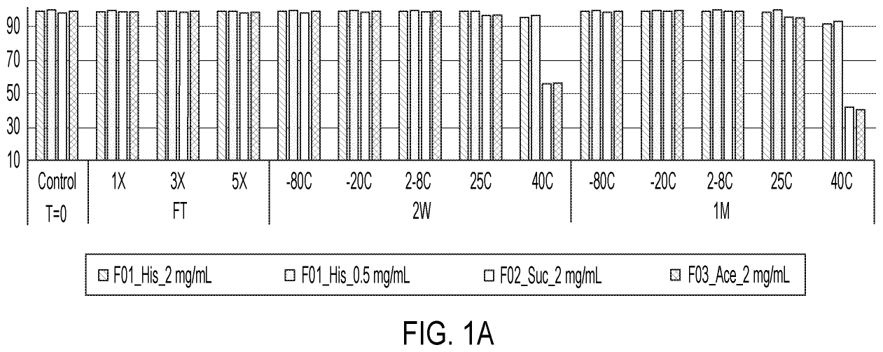

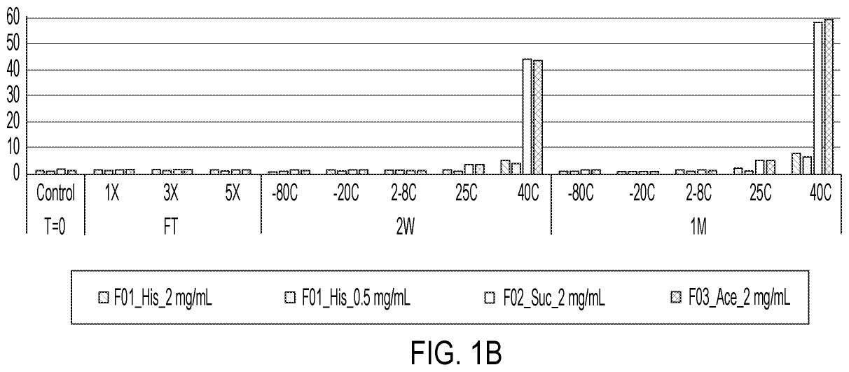

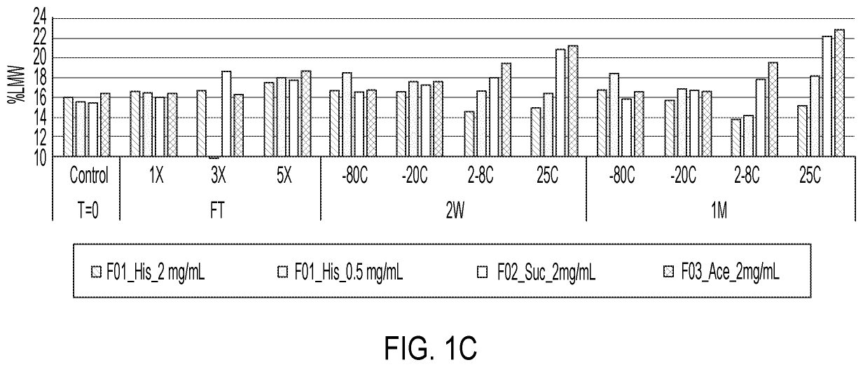

FIG. 1A provides the % monomer observed across the conditions tested. As described in FIG. 1A, Formulations F2 and F3 showed significant decreases of monomer levels at 40° C. after 2 weeks and 1 month.

FIG. 1B provides the sum of the % HMW and % LMW species observed across all conditions tested. Similar to the monomer results, Formulations F2 and F3 showed significant increases in aggregation at 40° C.

FIG. 1C provides the sum of the % HMW and % LMW species with the 40° C. results excluded so differences can be better viewed with a smaller range on the Y axis of the table. Overall, the Fla and F1b formulations provided consistent stability results.

### Capillary Electrophoresis Sodium Dodecyl Sulfate (CE-SDS)

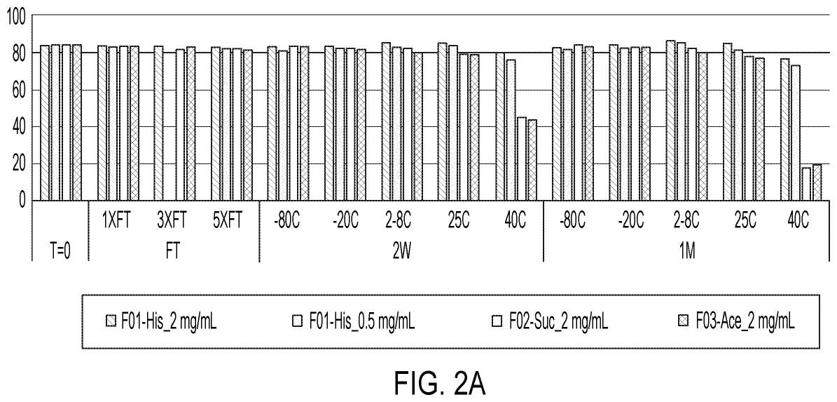

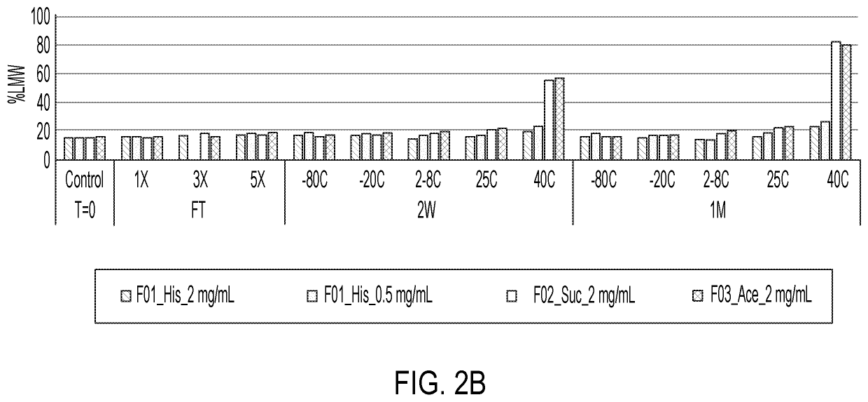

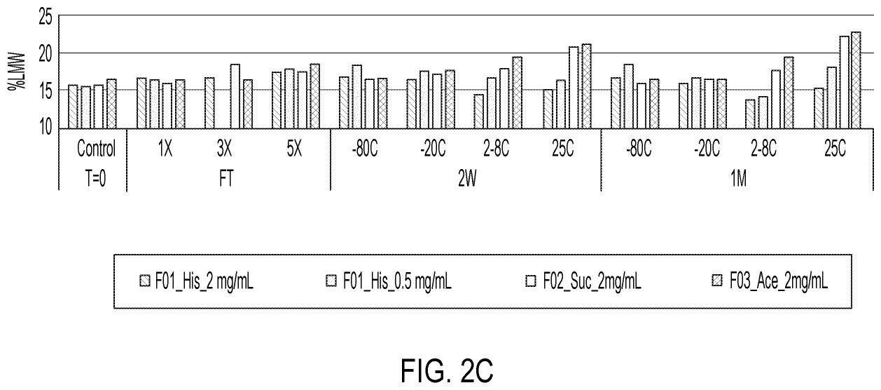

The percent of the main peak as analyzed by CE-SDS (represented as % purity) and the percent of low molecular weight species as analyzed by CE-SDS (represented as % impurity) was determined. FIG. 2A provides the % purity across the conditions tested. FIG. 2B provides the % impurity across all conditions tested. FIG. 2C provides the % impurity with the 40° C. results excluded.

No effect of F/T cycles on the purity of the CD45 ADC in any formulation was observed. After incubation at 40° C. for 2 weeks and 1 month, a substantial decrease in % Purity was observed with F2 and F3 formulations. Consequently, F2 and F3 showed higher % impurity than either of the F1a or F1b formulations. A similar trend between the CE-SDS and SEC was observed. This indicated greater stability of CD45 ADC in the F1a and F1b formulations versus F2 or F3.

### SUMMARY

Based on the concentration, turbidity, SEC, and CE-SDS assays, the F1a and F1b formulations (histidine, pH 6.0) showed greater stability over F2 (succinate, pH 4.2) and F3 (acetate, pH 4.2). The F1a and F1b formulations in histidine demonstrated similar stability profiles and no effect of F/T up to five cycles was observed. In conclusion, the 10 mM histidine pH 6, 10% sucrose, 50 μM sodium bisulfite, 3 mM methionine, 0.01% polysorbate-20 provided a stable formulation for the CD45 ADC.

### Example 2: Comparative Study of Anti-CD45 Antibody and Anti-CD45 ADC

A comparative study of the naked anti-CD45 (Ab5) and the anti-CD45 ADC (Ab5 conjugated to DGN549 via a linker) was performed. Results showed that each of these molecules, while related, acted differently under testing conditions. The description of the anti-CD45 ADC is provided above. The naked antibody used was the same as that in the ADC, i.e., Ab5 whose sequences are provided in the Sequence Table provided herein.

The anti-CD45 antibody was formulated in 10 mM histidine, 150 mM sucrose, pH 6 at an antibody concentration of 15.4 mg/mL. The anti-CD45 ADC was formulated in 10 mM histidine pH 6, 10% sucrose, 50 μM sodium bisulfite, 3 mM methionine, 0.01% polysorbate-20, pH 6.

The study assessed freeze thaw cycles, pH stress, light stress, and agitation.

Freeze/thaw study: For freeze/thaw, both the antibody alone and the ADC formulations were assessed over 7 freeze thaw cycles. Antibody samples were taken through 1, 3, 5 and 7 freeze thaw cycles between −70° C. and ambient. All samples were held at ambient to thaw for 1 hour±10 mins. Testing was carried out after the seventh cycle and samples held at 2-8° C. during testing. Results are provided in Table 4.

TABLE 4
Summary of results for freeze-thaw stress conditions for anti-CD45
antibody

| | | Freeze/Thaw Cycle | | | |
|---|---|---|---|---|---|
| ASSAY | | 1 | 3 | 5 | 7 |
| | Appearance | Clear | Clear | Clear | Clear |
| | | colourless | colourless | colourless | colourless |
| | | liquid, | liquid, | liquid, | liquid, |
| | | free from | free from | free from | free from |
| | | visible | visible | visible | visible |
| | | particles. | particles | particles | particles |
| | Turbidity | I | II | < II | < II |
| | Colour | < Y6 | < Y6 | < Y6 | < Y6 |
| Protein Concentration | | 15.7 | 15.8 | 15.8 | 15.9 |
| (ma/mL) | | | | | |
| Analysis by SEC | % HMWS | 0.8 | 0.8 | 0.8 | 0.8 |
| | % Main Peak | 98.0 | 98.0 | 98.0 | 98.0 |
| | % LMWS | 1.3 | 1.2 | 1.2 | 1.2 |
| Non-Reduced CE-SDS | % L | 2.52 | 2.50 | 2.50 | 2.49 |
| | % H | 0.00 | 0.00 | 0.00 | 0.00 |
| | % HL | 2.86 | 2.90 | 2.82 | 2.82 |
| | % HH | 0.39 | 0.47 | 0.39 | 0.50 |
| | % HHL | 3.25 | 3.33 | 3.26 | 3.35 |
| | % Intact | 90.68 | 90.37 | 90.55 | 90.36 |
| | % HMWS | 0.15 | 0.29 | 0.35 | 0.32 |
| | Total Impurities | 9.3 | 9.6 | 9.5 | 9.6 |
| Reduced | % HC and LC | 98.8 | N/R | N/R | N/R |
| CE-SDS | | | | | |
| | % Total | 1.6 | N/R | N/R | N/R |
| | Impurities | | | | |
| icIEF | % Acidic | 54.5 | 54.2 | 53.8 | 54.2 |
| | % Main | 31.6 | 31.2 | 31.9 | 31.1 |
| | % Basic | 13.9 | 14.7 | 14.4 | 14.7 |
| Sub- | ≤1.0 and <2.0 μm | 7004 | 20119 | 30606 | 39051 |
| visible | ≤2.0 and <5.0 μm | 1064 | 2197 | 5389 | 7575 |
| Particulates | ≤5.0 and <10.0 μm | 69 | 113 | 840 | 1076 |
| (Counts/ | ≤10 and <100 μm | 11 | 15 | 380 | 362 |
| mL) | ≤25 and <100 μm | 0 | 2 | 74 | 64 |
| | ≤1.0 and <100 μm | 8148 | 22444 | 37215 | 48063 |
| | Mean ECD (μm) | 1.48 | 1.41 | 1.85 | 1.78 |

ADC samples were taken through 1, 3, 5 and 7 freeze thaw cycles between −70° C. and ambient. All samples were held at ambient to thaw for 1 hour±10 mins. Testing was carried out after the seventh cycle and samples held at 2-8° C. during testing. Results are provided in Table 5.

TABLE 5
Summary of results for freeze-thaw stress conditions for anti-
CD45 ADC

| | | Freeze Thaw Cycle | | | |
|---|---|---|---|---|---|
| ASSAY | | 1 | 3 | 5 | 7 |
| Appearance | Appearance | Clear | Clear | Clear | Clear |
| | | colourless | colourless | colourless | colourless |
| | | liquid, | liquid, | liquid, | liquid, |
| | | free from | free from | free from | free from |
| | | visible | visible | visible | visible |
| | | particles. | particles | particles | particles |
| | Turbidity | I | II | < II | < II |
| | Colour | < Y7 | < Y7 | < Y7 | < Y7 |
| Protein Concentration (mg/ml) | | 2.0 | 2.0 | 2.0 | 2.0 |
| Drug to Antibody (M1337) Ratio | | 1.9 | 2.0 | 1.9 | 1.9 |
| (DAR) by UV | | | | | |
| Drug to Antibody (M1337) Ratio | | 1.9 | 1.9 | 1.8 | 1.9 |
| (DAR) by SEC | | | | | |
| CBA | | 110 | N/A | N/A | N/A |
| Analysis | % LMWS | 0.2 | 0.2 | 0.2 | 0.2 |
| by SEC | % Main Peak | 97.4 | 97.4 | 97.6 | 97.3 |
| | % HMWS | 2.4 | 2.5 | 2.3 | 2.5 |
| Non- | % L | 2.0 | 2.1 | 2.0 | 2.1 |
| Reduced | % H | 0.5 | 0.6 | 0.6 | 0.6 |
| CE-SOS | % HL | 7.4 | 7.5 | 9.2 | 7.4 |
| | % HH | 0.8 | 0.9 | 0.8 | 0.8 |
| | % HHL | 6.3 | 6.3 | 6.2 | 6.3 |
| | % Intact | 82.5 | 82.2 | 80.7 | 82.5 |
| | % HMWS | 0.2 | 0.3 | 0.4 | 0.2 |
| | Total Impurities | 17.5 | 17.8 | 19.3 | 17.5 |
| Reduced | % HC and LC | 97.0 | N/R | N/R | N/R |
| CE-SOS | % Total Impurities | 3.0 | N/R | N/R | N/R |
| icIEF | % Acidic | 43.6 | 47.7 | 46.8 | 48.1 |
| | % Main | 27.5 | 31.2 | 30.6 | 30.3 |
| | % Basic | 28.9 | 21.0 | 22.6 | 21.6 |
| Sub-visible | ≤1.0 and <2.0 μm | 15101 | 75575 | 74290 | 81852 |
| Particulates | ≤2.0 and <5.0 μm | 3497 | 23269 | 25142 | 29798 |
| | ≤5.0 and <10.0 μm | 246 | 553 | 691 | 1169 |
| | ≤10 and <100 μm | 85 | 49 | 95 | 70 |
| (Counts/mL) | ≤25 and <100 μm | 8 | 10 | 8 | 2 |
| | ≤1.0 and <100 μm | 18929 | 99446 | 100217 | 112889 |
| | Mean ECD (μm) | 1.68 | 1.66 | 1.71 | 1.75 |

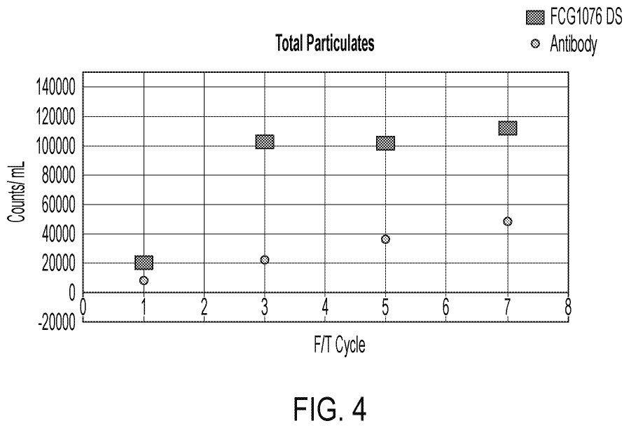

The only significant change in a key quality attributes were an increase in sub-visible particulates in both formulations. FIG. 4 graphically depicts the increase in subvisible particulates which were noted for both the antibody and the ADO over the seven FIT cycles.

Agitation study: The antibody alone and the ADO formulations were assessed over 5 days at 300 rpm.

Antibody (M1337; Ab5) samples were agitated at 300 rpm at 10° C. over five days. Sample vials were removed at T=3 days and T=5 days. Samples were held at 2-8° C. during testing. Results are provided in Table 6.

TABLE 6
Summary of results for agitation cycles for anti-CD45 antibody

| | | Agitation (Days) | | |
|---|---|---|---|---|
| ASSAY | | 0 | 3 | 5 |
| Appearance | Appearance | Clear | Clear | Clear |
| | | colourless | colourless | colourless |
| | | liquid, free | liquid, free | liquid, free |
| | | from visible | from visible | from visible |
| | | particles. | particles. | particles. |
| | Turbidity | I | < III | < III |
| | Colour | < Y6 | < Y6 | < Y6 |
| Analysis | % LMWS | 1.3 | 1.2 | 1.3 |
| by SEC | % Main Peak | 98.0 | 98.0 | 97.3 |
| | % HMWS | 0.8 | 0.8 | 1.6 |
| Sub-visible | ≤1.0 and <2.0 μm | 7004 | 33956 | 838718 |
| Particulates | ≤2.0 and <5.0 μm | 1064 | 6326 | 141894 |
| (Counts/mL) | ≤5.0 and <10.0 μm | 69 | 1405 | 29062 |
| | ≤10 and <100 μm | 11 | 730 | 9721 |
| | ≤25 and <100 μm | 0 | 219 | 827 |
| | ≤1.0 and <100 μm | 8148 | 42417 | 1019395 |
| | Mean ECD (μm) | 1.48 | 2.16 | 1.76 |

ADO samples were agitated at 300 rpm at 10° C. over five days. Sample vials were removed at T=3 days and T=5 days. Samples were held at 2-8° C. during testing.

TABLE 7
Summary of results for agitation cycles for anti-CD45 ADC

| | | Agitation (Days) | | |
|---|---|---|---|---|
| ASSAY | | 0 | 3 | 5 |
| Appearance | Appearance | Clear | Clear | Clear |
| | | colourless | colourless | colourless |
| | | liquid, free | liquid, free | liquid, free |
| | | from visible | from visible | from visible |
| | | particles. | particles. | particles. |
| | Turbidity | < II | II | < II |
| | Colour | < Y7 | < Y7 | < Y7 |
| Drug to Antibody (M1337) | | 1.9 | 1.8 | 1.8 |
| Ratio | | | | |
| (DAR) by SEC | | | | |
| Analysis | % LMWS | 0.2 | 0.2 | 0.2 |
| by SEC | % Main Peak | 97.4 | 93.4 | 93.7 |
| | % HMWS | 2.4 | 6.4 | 6.1 |
| Sub-visible | ≤1.0 and <2.0 μm | 81852 | 16761 | 3664 |
| Particulates | ≤2.0 and <5.0 μm | 29798 | 4728 | 1130 |
| (Counts/mL) | ≤5.0 and <10.0 μm | 1169 | 201 | 82 |
| | ≤10 and <100 μm | 70 | 56 | 7 |
| | ≤25 and <100 μm | 2 | 2 | 0 |
| | ≤1.0 and <100 μm | 112889 | 21746 | 4882 |
| | Mean ECD (μm) | 1.75 | 1.67 | 1.75 |

The results provided in Table 7 showed a general increase in turbidity, sub-visible particulates and aggregation by SEC were noted for both formulations. All other quality attributes remained the same.

General increase in turbidity noted for both the antibody and the ADC. The antibody showed the greatest increase with T=5 days equivalent to turbidity reference standard Ill. A general increase in high molecular weight species was noted for both the antibody and the ADC. The ADC showed an increase in all high molecular weight species, while the antibody showed an increase in only one type of the HMWs.

pH stress—low pH 3.5: The antibody alone and the ADC formulations were assessed over 3 days. More specifically, 60 mLs of ADC and antibody were thawed at room temperature and mixed gently by inversion. Each 60 mL aliquot of antibody and ADC was then split into 2×30 mL aliquots. One 30 mL aliquot each of antibody and ADC was pH adjusted to pH 3.5 using 0.5M HCl. One 30 mL aliquot each of antibody and ADO was pH adjusted to pH 8.5 using 0.5M NaOH.

For T0 (time 0), 8 mLs of each sample point was placed in a polycarbonate bottle and pH adjusted to pH 6.0±0.2. All samples were stored at room temperature/for T=1 and T=3 days samples were quenched with either 0.5M NaOH or 0.5M HCl as appropriate. Results are provided below in Tables 8 and 9 of the analysis from the low pH stress test.

TABLE 8
Summary of results for F/T 1 and pH low stress time points for anti-CD45
antibody

| | | Low pH Stress pH 3.5 | | | |
|---|---|---|---|---|---|
| ASSAY | | F/T 1 | TO | T1 | T3 |
| Appearance | Appearance | Clear | Clear | Clear | Clear |
| | | colourless | colourless | colourless | colourless |
| | | liquid, | liquid, free | liquid, free | liquid, free |
| | | free from | from visible | from visible | from visible |
| | | visible | particles. | particles | particles |
| | | particles. | | | |
| | Turbidity | I | < II | II | < III |
| | Colour | < Y6 | < Y6 | < Y6 | < Y6 |
| Protein Concentration (ma/mL) | | 15.7 | 15.4 | 15.3 | 15.2 |
| Analysis | % HMWS | 0.8 | 2.0 | 1.8 | 1.8 |
| by SEC | % Main Peak | 98.0 | 96.9 | 96.9 | 96.8 |
| | % LMWS | 1.3 | 1.2 | 1.2 | 1.2 |
| Non- | % L | 2.5 | 2.4 | 2.5 | 2.6 |
| Reduced | % H | 0.0 | 0.0 | 0.0 | 0.0 |
| CE-SOS | % HL | 2.9 | 2.8 | 2.8 | 2.7 |
| | % HH | 0.4 | 0.4 | 0.4 | 0.6 |
| | % HHL | 3.3 | 3.2 | 3.3 | 3.6 |
| | % Intact | 90.7 | 90.7 | 90.5 | 90.0 |
| | % HMWS | 0.2 | 0.4 | 0.4 | 0.4 |
| | Total Impurities | 9.3 | 9.3 | 9.5 | 10.0 |
| Reduced | % HC and LC | 98.8 | 98.7 | 98.8 | 98.0 |
| CE-SOS | % Total Impurities | 1.6 | 1.7 | 1.6 | 2.4 |
| icIEF | % Acidic | 54.5 | 54.3 | 54.3 | 54.3 |
| | % Main | 31.6 | 31.8 | 31.7 | 31.0 |
| | % Basic | 13.9 | 13.9 | 14.0 | 14.7 |

TABLE 9
Summary of results for F/T 1 and pH low stress time points for anti-CD45
ADC

| | | Low pH Stress pH 3.5 | | | |
|---|---|---|---|---|---|
| ASSAY | | F/T 1 | TO | T1 | T3 |
| Appearance | Appearance | Clear | Clear | Clear | Clear |
| | | colourless | colourless | colourless | colourless |
| | | liquid, free | liquid, | liquid, free | liquid, free |
| | | from visible | free from | from visible | from visible |
| | | particles. | visible | particles. | particles. |
| | | | particles. | | |
| | Turbidity | < II | < III | < III | < III |
| | Colour | < Y7 | < Y7 | < Y7 | < Y7 |
| Protein Concentration (ma/mL) | | 2.0 | 2.0 | 1.9 | 2.0 |
| Drug to Antibody (M1337) Ratio | | 1.9 | 2.0 | 2.0 | 1.9 |
| (DAR) by UV | | | | | |
| Drug to Antibody (M1337) Ratio | | 1.9 | 1.9 | 1.9 | 1.9 |
| (DAR) by SEC | | | | | |
| CBA | | 110 | 105 | 136 | 144 |
| Analysis | % LMWS | 0.2 | 0.2 | 0.2 | 0.3 |
| by SEC | % Main Peak | 97.4 | 95.1 | 91.1 | 78.8 |
| | % HMWS | 2.4 | 4.7 | 8.7 | 20.9 |
| Non- | % L | 2.0 | 2.1 | 2.1 | 2.2 |
| Reduced | % H | 0.5 | 0.6 | 0.6 | 0.6 |
| CE-SOS | % HL | 7.4 | 7.8 | 8.4 | 9.0 |
| | % HH | 0.8 | 0.9 | 0.9 | 1.0 |
| | % HHL | 6.3 | 6.4 | 6.6 | 6.9 |
| | % Intact | 82.5 | 81.7 | 80.7 | 79.6 |
| | % HMWS | 0.2 | 0.5 | 0.5 | 0.6 |
| | Total Impurities | 17.5 | 18.3 | 19.3 | 20.4 |
| Reduced | % HC and LC | 97.0 | 96.9 | 96.7 | 96.2 |
| CE-SOS | % Total Impurities | 3.0 | 3.1 | 3.3 | 3.8 |
| icIEF | % Acidic | 43.6 | 47.9 | 47.3 | 38.2 |
| | % Main | 27.5 | 29.8 | 28.5 | 28.9 |
| | % Basic | 28.9 | 22.4 | 24.2 | 33.2 |

In sum, initial change noted upon pH adjust at T=0 days when compared to initial F/T with no pH adjust for purity by SEC, % intact by NR-CE-SDS and % main peak purity by icIEF. Intact % purity by non-reduced CE-SOS, % HC and LC by reduced CE-SOS, % main peak purity by SEC, % purity by charge heterogeneity and potency by CBA all showed a change throughout by the low pH stress study.

Intact % purity by non-reduced CE-SDS, % heacy chain (HC) and light chain (LC) by reduced CE-SDS, % main peak purity by SEC, % purity by charge heterogeneity and potency by CBA all showed no change throughout the low pH stress study. Drug to antibody ratio (DAR) remained consisted by SEC and there was no evidence of loss of drug linker noted from peptide mapping data.

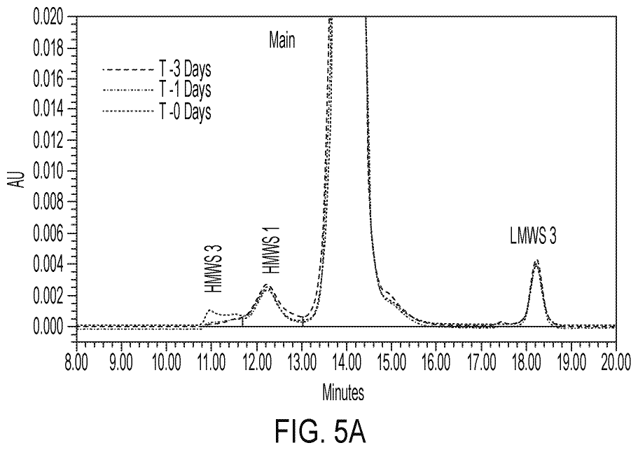

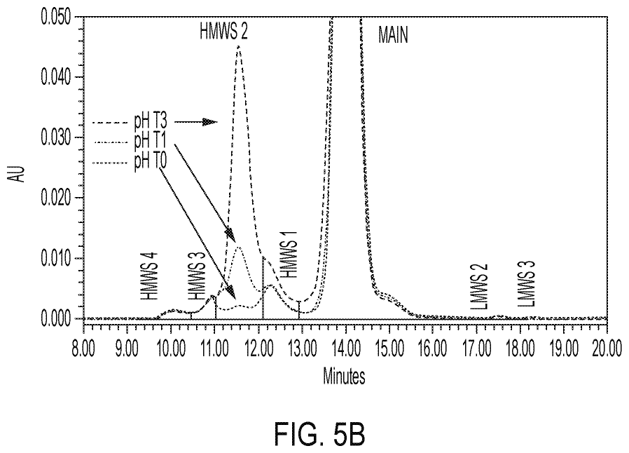

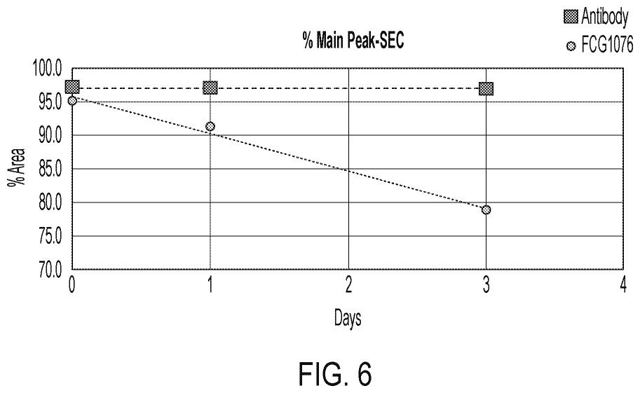

Antibody aggregate levels showed no significant increase at pH 3.4, while the ADC showed a significant increase in HMW levels (specifically species 2), as shown in FIGS. 5A and B. FIG. 6 describes antibody and ADC % main peak as determined by SEC, where the ADC showed a more rapid degradation than the antibody in the low pH stress test.

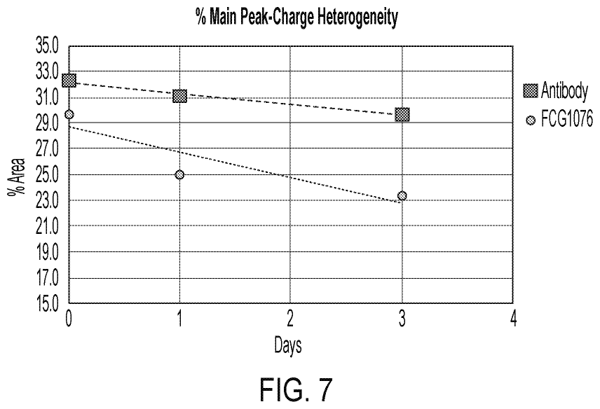

pH stress study—high pH 8.5: Main quality attributes affected by high pH was % main peak purity by icIEF. The antibody alone and the ADC formulations both showed decrease in main peak purity with the ADC formulations showing a faster rate of degradation, as described in FIG. 7.

Light stress study: Main key quality attributes affected by light stress were a decrease in % main peak by SEC. An increase in oxidation was noted, and a loss of drug linker was suggested by reduced and non-reduced peptide mapping data. The DAR by SEC showed a decrease in DAR by 0.5, from 1.9 to 1.4. Further subunit analysis showed degradation of the drug linker with loss of sulfonation and PBD groups.

### Example 3: Sulfonation Reaction

The sulfonation reaction was used to introduce a sulfite group to the imine of DGN549-C, increasing its solubility and reactivity.

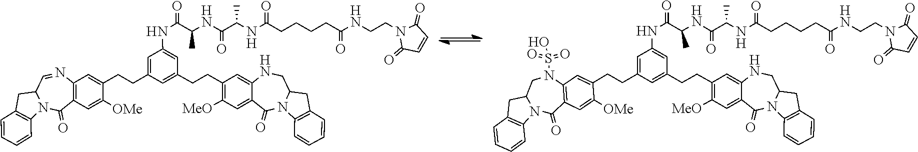 [SMILES: COc1cc2c(cc1CCc1cc(CCc3cc4c(cc3OC)C(=O)N3c5ccccc5CC3CN4)cc(NC(=O)[C@H](C)NC(=O)[C@H](C)NC(=O)CCCCC(=O)NCCN3C(=O)C=CC3=O)c1)N=CC1Cc3ccccc3N1C2=O ; COc1cc2c(cc1CCc1cc(CCc3cc4c(cc3OC)C(=O)N3c5ccccc5CC3CN4S(=O)(=O)O)cc(NC(=O)[C@H](C)NC(=O)[C@H](C)NC(=O)CCCCC(=O)NCCN3C(=O)C=CC3=O)c1)NCC1Cc3ccccc3N1C2=O]

DGN549-C was sulfonated at 25° C. for 3 hours in 50 mM succinate pH 3.3 with 50% (v/v) DMA with a 1.4 molar excess of bisulfite, varying the concentration to 1, 3 and 5 mM. The concentration of DGN549-C in the sulfonation reaction had a very strong effect on the levels and type of sulfonation. Increasing the concentration above 1 mM to 3 and 5 mM increased the overall reactivity of the reaction, resulting in greater sulfonation of the maleimide, sharply reducing the availability of DGN549-C for conjugation.

A study to optimize the sulfonation reaction was performed. The sulfonation reaction parameters were optimized according to the conditions below.

### Optimized Process Parameter Conditions for Sulfonation Reaction

| | | Recommended |
|---|---|---|
| | | Conditions for |
| | | Optimized |
| Process Parameter | Target Condition | Product Quality |
| DGN Concentration | 1.0 | 1.0 |
| (mM) | | |
| Sodium Bisulfite Excess | 1.4 | 1.5 |
| DMA % (v/v) | 50 | 50 |
| Reaction Time (hr) | 4.5 | 3 |
| Reaction Temperature | 25 | 23 |
| (° C.) | | |
| Succinate Buffer pH | 3.3 | 3.3 |

f

TABLE 10
Sequence Table

| SEQ ID | | |
|---|---|---|
| NO: | Description | Sequence |
| 1 | Ab5 Heavy Chain | EVQLVESGGGLVQPGGSLRLSCAASGFTFEAYSMNWVRQA |
| | Variable Region | PGKGLEWVSYISLSGATIHYADSVKGRFTISRDNAKNSLYLQ |
| | (CDRs bolded) | MNSLRAEDTAVYYCARGGQYYYTSSDYGEVAFDIWGQGTM |
| | | VTVSS |
| 2 | Ab5-HC CDR1 | FTFEAYSMN |
| 3 | Ab5-HC CDR2 | YISLSGATIHYADSVKG |
| 4 | Ab5-HC CDR3 | ARGGQYYYTSSDYGEVAFDI |
| 5 | Ab5 Light Chain | DIVMTQSPLSLPVTPGEPASISCRSSQSLVSSGYNYLDWYLQ |
| | Variable Region | KPGQSPQLLIYFGSSRASGVPDRFSGSGSGTDFTLKISRVEA |
| | (CDRs) Bolded | EDVGVYYCMQRRRTPWSFGGGTKVEIK |
| 6 | Ab5-LC CDR1 | RSSQSLVSSGYNYLD |
| 7 | Ab5-LC CDR2 | FGSSRAS |
| 8 | Ab5-LC CDR3 | MQRRRTPWS |
| 9 | Ab5 Heavy Chain | EVQLVESGGGLVQPGGSLRLSCAASGFTFEAYSMNWVRQA |
| | (CDRs in bold; | PGKGLEWVSYISLSGATIHYADSVKGRFTISRDNAKNSLYLQ |
| | Constant region | MNSLRAEDTAVYYCARGGQYYYTSSDYGEVAFDIWGQGTM |
| | underlined; | VTVSSASTKGPSVFPLAPSSKSTSGGTAALGCLVKDYFPEPV |
| | engineered | TVSWNSGALTSGVHTFPAVLQSSGLYSLSSVVTVPSSSLGTQ |
| | cysteine residue | TYICNVNHKPSNTKVDKKVEPKSCDKTHTCPPCPAPEAAGGP |
| | for conjugation | SVFLFPPKPKDTLMISRTPEVTCVVVCVSHEDPEVKFNWYVD |
| | site to IGN in | GVEVHNAKTKPREEQYNSTYRVVSVLTVLHQDWLNGKEYKC |
| | bold/italics) | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 10 | Ab5 Light Chain | DIVMTQSPLSLPVTPGEPASISCRSSQSLVSSGYNYLDWYLQ |
| | (CDRs in bold; | KPGQSPQLLIYFGSSRASGVPDRFSGSGSGTDFTLKISRVEA |
| | Constant region | EDVGVYYCMQRRRTPWSFGGGTKVEIKRTVAAPSVFIFPPS |
| | underlined) | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 11 | Ab5 Heavy Chain | ggtaccgccaccatggatctgctgcacaagaacatgaagcacctgtggttctttctgct |
| | DNA nucleotide | gctggtggccgctcctagatgggtgctgtctgaagtgcagctggtggaatctggcggag |
| | sequence (coding | gattggttcagcctggcggctctctgagactgtcttgtgctgcctctggcttcaccttcgag |
| | region underlined; | gcctactccatgaactgggtccgacaggctcctggcaaaggactggaatgggtgtcct |
| | restriction site in | acatctctctgtctggcgccaccatccactacgccgattctgtgaagggcagattcacc |
| | bold; Kozak | atcagccgggacaacgccaagaactccctgtacctgcagatgaacagcctgagagc |
| | sequence in | cgaggacaccgccgtgtactattgtgctagaggcggccagtactactacacctcctctg |
| | italics) | attacggcgaggtggccttcgatatctggggccagggaacaatggtcaccgtgtcctct |
| | | gcttccaccaagggaccttccgtgtttcctctggctccttccagcaagtctacctctggtgg |
| | | aaccgctgctctgggctgcctggtcaaggattactttcctgagcctgtgaccgtgtcttgq |
| | | aactctggtgctctgacctccggcgtgcacacatttccagctgtgctgcagtcctccggc |
| | | ctgtactctctgtcctctgtcgtgaccgtgccttctagctctctgggcacccagacctacat |
| | | ctgcaacgtgaaccacaagccttccaacaccaaggtggacaagaaggtggaaccc |
| | | aagtcctgcgacaagacccacacctgtcctccatgtcctgctccagaagctgctggcq |
| | | gaccctctgtgttcctgtttccacctaagcctaaggacaccctgatgatctctcggacccc |
| | | tgaagtgacctgcgtqgtcgtgtgtgtgtctcacgaggacccagaagtgaagttcaatt |
| | | ggtacgtggacggcgtggaagtgcacaatgccaagaccaagcctagagaggaaca |
| | | gtacaactccacctacagagtggtgtccgtgctgaccgtgctgcaccaggattggctga |
| | | acggcaaagagtacaagtgcaaggtgtccaacaaggccctgcctgctcctatcgaa |
| | | aagaccatctccaaggctaagggccagcctcgggaacctcaggtttacacactgcct |
| | | ccatctcgggacgagctgaccaagaatcaggtgtccctgacctgcctcgtgaagggct |
| | | tctacccttccgatatcgccgtggaatgggagtctaacggccagccagagaacaacta |
| | | caagacaacccctcctgtgctggactccgacggctcattcttcctgtactccaagctgac |
| | | agtggacaagtctcggtqqcagcagggcaacqtgttctcctgttctgtgatgcacgagg |
| | | ccctgcacaacgcctacacacagaagtccctgtctctgtcccctggcaagtgatagaa |
| | | gctt |
| 12 | Ab5 Light Chain | ggatccgccaccatggatctgctgcacaagaacatgaagcacctgtggttctttctgct |
| | DNA nucleotide | gctggtggccgctcctagatgggtgctgtctgacatcgtgatgacccagtctccactgag |
| | sequence (coding | cctgcctgttacacctggcgagcctgccagcatctcctgcagatcttcccagtctctggtg |
| | region underlined; | tcctccggctacaactacctggactggtatctgcagaagcccggccagtctcctcagct |
| | restriction site in | gctgatctactttggctcctccagagcctctggcgtgcccgatagattttctggctctggct |
| | bold; Kozak | ccggcaccgacttcaccctgaagatctctagagtggaagccgaggacgtgggcgtgt |
| | sequence in | actactgtatgcagcggagaagaaccccttggtctttcggcggaggcaccaaggtgg |
| | italics) | aaatcaagagaaccgtggctgccccttccgtgttcatcttcccaccatctgacgagcag |
| | | ctgaagtctggcaccgcttctgtcgtgtgcctgctgaacaacttctaccctcgggaagcc |
| | | aaggtgcagtggaaggtggacaatgccctgcagtccggcaactcccaagagtctgtg |
| | | accgagcaggactccaaggactctacctacagcctgtcctccacactgaccctgtcta |
| | | aggccgactacgagaagcacaaggtgtacgcctgtgaagtgacccaccagggact |
| | | gtctagccccgtgaccaagtctttcaacagaggcgagtgctgatagctcgag |
| 13 | Ab1 Heavy Chain | QVQLVESGGGLVKPGGSLRLSCAASGFTFSSYSMNWVRQA |
| | Variable Region | PGKGLEWVSYISSSSSTIYYADSVKGRFTISRDNAKNSLYLQ |
| | (CDRs bolded) | MNSLRAEDTAVYYCARGGQYYYDSSRYGEVAFDIWGQGTM |
| | | VTVSS |
| 14 | Ab1-HC CDR1 | FTFSSYSMN |
| 15 | Ab1-HC CDR2 | YISSSSSTIYYADSVKG |
| 16 | Ab1-HC CDR3 | ARGGQYYYDSSRYGEVAFDI |
| 17 | Ab1 Light Chain | DIVMTQSPLSLPVTPGEPASISCRSSQSLLHSNGYNYLDWYL |
| | Variable Region | |
| | (CDRs bolded) | QKPGQSPQLLIYLGSNRASGVPDRFSGSGSGTDFTLKISRVE |
| | | AEDVGVYYCMQRRRTPPFTFGGGTKVEIK |
| 18 | Ab1-LC CDR1 | RSSQSLLHSNGYNYLD |
| 19 | Ab1-LC CDR2 | LGSNRAS |
| 20 | Ab1-LC CDR3 | MQRRRTPPFT |
| 21 | Ab1 Heavy Chain | QVQLVESGGGLVKPGGSLRLSCAASGFTFSSYSMNWVRQA |
| | (CDRs in bold; | PGKGLEWVSYISSSSSTIYYADSVKGRFTISRDNAKNSLYLQ |
| | Constant region | MNSLRAEDTAVYYCARGGQYYYDSSRYGEVAFDIWGQGTM |
| | underlined; | VTVSSASTKGPSVFPLAPSSKSTSGGTAALGCLVKDYFPEPV |
| | D265C.LALA.H435A) | TVSWNSGALTSGVHTFPAVLQSSGLYSLSSVVTVPSSSLGTQ |
| | | TYICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 22 | Ab1 Light Chain | DIVMTQSPLSLPVTPGEPASISCRSSQSLLHSNGYNYLDWYL |
| | (CDRs in bold; | QKPGQSPQLLIYLGSNRASGVPDRFSGSGSGTDFTLKISRVE |
| | Constant region | AEDVGVYYCMQRRRTPPFTFGGGTKVEIKRTVAAPSVFIFPP |
| | underlined) | SDEQLKSGTASVVCLLNNFYPREAKVQWKVDNALQSGNSQE |
| | | SVTEQDSKDSTYSLSSTLTLSKADYEKHKVYACEVTHQGLSS |
| | | PVTKSFNRGEC |
| 23 | Ab2 Heavy Chain | EVQLVESGGGLVQPGGSLRLSCAASGFTFEAYSMNWVRQA |
| | Variable Region | PGKGLEWVSYISLSGATIHYADSVKGRFTISRDNAKNSLYLQ |
| | (CDRs bolded) | MNSLRAEDTAVYYCARGGQYYYDSSDYGEVAFDIWGQGTM |
| | | VTVSS |
| 24 | Ab2-HC CDR1 | FTFEAYSMN |
| 25 | Ab2-HC CDR2 | YISLSGATIHYADSVKG |
| 26 | Ab2-HC CDR3 | ARGGQYYYDSSDYGEVAFDI |
| 27 | Ab2 Light Chain | DIVMTQSPLSLPVTPGEPASISCRSSQSLVSNGYNYLDWYLQ |
| | Variable Region | KPGQSPQLLIYFGSSRASGVPDRFSGSGSGTDFTLKISRVEA |
| | (CDRs bolded) | EDVGVYYCMQRRRTPWSFGGGTKVEIK |
| 28 | Ab2-LC CDR1 | RSSQSLVSNGYNYLD |
| 29 | Ab2-LC CDR2 | FGSSRAS |
| 30 | Ab2-LC CDR3 | MQRRRTPWS |
| 31 | Ab2 Heavy Chain | EVQLVESGGGLVQPGGSLRLSCAASGFTFEAYSMNWVRQA |
| | (CDRs in bold; | PGKGLEWVSYISLSGATIHYADSVKGRFTISRDNAKNSLYLQ |
| | Constant region | MNSLRAEDTAVYYCARGGQYYYDSSDYGEVAFDIWGQGTM |
| | underlined; | VTVSSASTKGPSVFPLAPSSKSTSGGTAALGCLVKDYFPEPV |
| | D265C.LALA.H435A) | TVSWNSGALTSGVHTFPAVLQSSGLYSLSSVVTVPSSSLGTQ |
| | | TYICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 32 | Ab2 Light Chain | DIVMTQSPLSLPVTPGEPASISCRSSQSLVSNGYNYLDWYLQ |
| | (CDRs in bold; | KPGQSPQLLIYFGSSRASGVPDRFSGSGSGTDFTLKISRVEA |
| | Constant region | EDVGVYYCMQRRRTPWSFGGGTKVEIKRTVAAPSVFIFPPS |
| | underlined) | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 33 | Ab3 Heavy Chain | QVQLVESGGGLVKPGGSLRLSCAASGFTFGGYSMNWVRQA |
| | Variable Region | PGKGLEWVSYISISGATITYADSVKGRFTISRDNAKNSLYLQM |
| | (CDRs bolded) | NSLRAEDTAVYYCARGGQYYYDSSDYGEVAFDIWGQGTMV |
| | | TVSS |
| 34 | Ab3-HC CDR1 | FTFGGYSMN |
| 35 | Ab3-HC CDR2 | YISISGATITYADSVKG |
| 36 | Ab3-HC CDR3 | ARGGQYYYDSSDYGEVAFDI |
| 37 | Ab3 Light Chain | DIVMTQSPLSLPVTPGEPASISCRSSQSLVSNGYNYLDWYLQ |
| | Variable Region | KPGQSPQLLIYFGSSRASGVPDRFSGSGSGTDFTLKISRVEA |
| | (CDRs bolded) | EDVGVYYCMQRRRTPPFTFGGGTKVEIK |
| 38 | Ab3-LC CDR1 | RSSQSLVSNGYNYLD |
| 39 | Ab3-LC CDR2 | FGSSRAS |
| 40 | Ab3-LC CDR3 | MQRRRTPPFT |
| 41 | Ab3 Heavy Chain | QVQLVESGGGLVKPGGSLRLSCAASGFTFGGYSMNWVRQA |
| | (CDRs in bold; | PGKGLEWVSYISISGATITYADSVKGRFTISRDNAKNSLYLQM |
| | Constant region | NSLRAEDTAVYYCARGGQYYYDSSDYGEVAFDIWGQGTMV |
| | underlined; | TVSSASTKGPSVFPLAPSSKSTSGGTAALGCLVKDYFPEPVT |
| | D265C.LALA.H435A) | VSWNSGALTSGVHTFPAVLQSSGLYSLSSVVTVPSSSLGTQT |
| | | YICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 42 | Ab3 Light Chain | DIVMTQSPLSLPVTPGEPASISCRSSQSLVSNGYNYLDWYLQ |
| | (CDRs in bold; | KPGQSPQLLIYFGSSRASGVPDRFSGSGSGTDFTLKISRVEA |
| | Constant region | EDVGVYYCMQRRRTPPFTFGGGTKVEIKRTVAAPSVFIFPPS |
| | underlined) | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 43 | Ab4 Heavy Chain | EVQLVESGGGLVQPGGSLRLSCAASGFTFEAYSMNWVRQA |
| | Variable Region | PGKGLEWVSYISLSGATIHYADSVKGRFTISRDNAKNSLYLQ |
| | (CDRs bolded) | MNSLRAEDTAVYYCARGGQYYYTSSDYGEVAFDIWGQGTM |
| | | VTVSS |
| 44 | Ab4-HC CDR1 | FTFEAYSMN |
| 45 | Ab4-HC CDR2 | YISLSGATIHYADSVKG |
| 46 | Ab4-HC CDR3 | ARGGQYYYTSSDYGEVAFDI |
| 47 | Ab4 Light Chain | DIVMTQSPLSLPVTPGEPASISCRSSQSLVSNGYNYLDWYLQ |
| | Variable Region | KPGQSPQLLIYFGSSRASGVPDRFSGSGSGTDFTLKISRVEA |
| | (CDRs bolded) | EDVGVYYCMQRRRTPWSFGGGTKVEIK |
| 48 | Ab4-LC CDR1 | RSSQSLVSNGYNYLD |
| 49 | Ab4-LC CDR2 | FGSSRAS |
| 50 | Ab4-LC CDR3 | MQRRRTPWS |
| 51 | Ab4 Heavy Chain | EVQLVESGGGLVQPGGSLRLSCAASGFTFEAYSMNWVRQA |
| | (CDRs in bold; | PGKGLEWVSYISLSGATIHYADSVKGRFTISRDNAKNSLYLQ |
| | Constant region | MNSLRAEDTAVYYCARGGQYYYTSSDYGEVAFDIWGQGTM |
| | underlined; | VTVSSASTKGPSVFPLAPSSKSTSGGTAALGCLVKDYFPEPV |
| | D265C.LALA.H435A) | TVSWNSGALTSGVHTFPAVLQSSGLYSLSSVVTVPSSSLGTQ |
| | | TYICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 52 | Ab4 Light Chain | DIVMTQSPLSLPVTPGEPASISCRSSQSLVSNGYNYLDWYLQ |
| | (CDRs in bold; | KPGQSPQLLIYFGSSRASGVPDRFSGSGSGTDFTLKISRVEA |
| | Constant region | EDVGVYYCMQRRRTPWSFGGGTKVEIKRTVAAPSVFIFPPS |
| | underlined) | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |
| 53 | Ab6 Heavy Chain | EVQLVESGGGLVQPGGSLRLSCAASGFTFEAYSMNWVRQA |
| | Variable Region | PGKGLEWVSYISLSGATIHYADSVKGRFTISRDNAKNSLYLQ |
| | (CDRs bolded) | MNSLRAEDTAVYYCARGGQYYYTSSDYGEVAFDIWGQGTLV |
| | | TVSS |
| 54 | Ab6-HC CDR1 | FTFEAYSMN |
| 55 | Ab6-HC CDR2 | YISLSGATIHYADSVKG |
| 56 | Ab6-HC CDR3 | ARGGQYYYTSSDYGEVAFDI |
| 57 | Ab6 Light Chain | DIVLTQSPLSLPVTPGEPASISCRSSQSLVSSGYNYLDWYLQ |
| | Variable Region | KPGQSPQLLIYFGSSRASGVPDRFSGSGSGTDFTLKISRVEA |
| | (CDRs bolded) | EDVGVYYCMQRRRTPWSFGGGTKVEIK |
| 58 | Ab6-LC CDR1 | RSSQSLVSSGYNYLD |
| 59 | Ab6-LC CDR2 | FGSSRAS |
| 60 | Ab6-LC CDR3 | MQRRRTPWS |
| 61 | Ab6 Heavy Chain | EVQLVESGGGLVQPGGSLRLSCAASGFTFEAYSMNWVRQA |
| | (CDRs in bold; | PGKGLEWVSYISLSGATIHYADSVKGRFTISRDNAKNSLYLQ |
| | Constant region | MNSLRAEDTAVYYCARGGQYYYTSSDYGEVAFDIWGQGTLV |
| | underlined; | TVSSASTKGPSVFPLAPSSKSTSGGTAALGCLVKDYFPEPVT |
| | D265C.LALA.H435A) | VSWNSGALTSGVHTFPAVLQSSGLYSLSSVVTVPSSSLGTQT |
| | | YICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 62 | Ab6 Light Chain | DIVLTQSPLSLPVTPGEPASISCRSSQSLVSSGYNYLDWYLQ |
| | (CDRs in bold; | KPGQSPQLLIYFGSSRASGVPDRFSGSGSGTDFTLKISRVEA |
| | | EDVGVYYCMQRRRTPWSFGGGTKVEIKRTVAAPSVFIFPPS |
| | Constant region | DEQLKSGTASVVCLLNNFYPREAKVQWKVDNALQSGNSQES |
| | underlined) | VTEQDSKDSTYSLSSTLTLSKADYEKHKVYACEVTHQGLSSP |
| | | VTKSFNRGEC |
| 63 | Ab7 Heavy Chain | QVQLVESGGGLVKPGGSLRLSCAASGFTFGGYSMNWVRQA |
| | Variable Region | PGKGLEWVSYISISGATITYADSVKGRFTISRDNAKNSLYLQM |
| | (CDRs bolded) | NSLRAEDTAVYYCARGGQYYYDSSDYGEVAFDIWGQGTMV |
| | | TVSS |
| 64 | Ab7-HC CDR1 | FTFGGYSMN |
| 65 | Ab7-HC CDR2 | YISISGATITYADSVKG |
| 66 | Ab7-HC CDR3 | ARGGQYYYDSSDYGEVAFDI |
| 67 | Ab7 Light Chain | DIVMTQSPLSLPVTPGEPASISCRSSQSLVSSGYNYLDWYLQ |
| | Variable Region | KPGQSPQLLIYFGSSRASGVPDRFSGSGSGTDFTLKISRVEA |
| | (CDRs bolded) | EDVGVYYCMQRRRTPPFTFGGGTKVEIK |
| 68 | Ab7-LC CDR1 | RSSQSLVSSGYNYLD |
| 69 | Ab7-LC CDR2 | FGSSRAS |
| 70 | Ab7-LC CDR3 | MQRRRTPPFT |
| 71 | Ab7 Heavy Chain | QVQLVESGGGLVKPGGSLRLSCAASGFTFGGYSMNWVRQA |
| | (CDRs in bold; | PGKGLEWVSYISISGATITYADSVKGRFTISRDNAKNSLYLQM |
| | Constant region | NSLRAEDTAVYYCARGGQYYYDSSDYGEVAFDIWGQGTMV |
| | underlined; | TVSSASTKGPSVFPLAPSSKSTSGGTAALGCLVKDYFPEPVT |
| | D265C.LALA.H435A) | VSWNSGALTSGVHTFPAVLQSSGLYSLSSVVTVPSSSLGTQT |
| | | YICNVNHKPSNTKVDKKVEPKSCDKTHTCPPCPAPEAAGGP |
| | | SVFLFPPKPKDTLMISRTPEVTCVVVCVSHEDPEVKFNWYVD |
| | | GVEVHNAKTKPREEQYNSTYRVVSVLTVLHQDWLNGKEYKC |
| | | KVSNKALPAPIEKTISKAKGQPREPQVYTLPPSRDELTKNQVS |
| | | LTCLVKGFYPSDIAVEWESNGQPENNYKTTPPVLDSDGSFFL |
| | | YSKLTVDKSRWQQGNVFSCSVMHEALHNAYTQKSLSLSPGK |
| 72 | Ab7 Light Chain | DIVMTQSPLSLPVTPGEPASISCRSSQSLVSSGYNYLDWYLQ |
| | (CDRs in bold; | KPGQSPQLLIYFGSSRASGVPDRFSGSGSGTDFTLKISRVEA |
| | Constant region | EDVGVYYCMQRRRTPPFTFGGGTKVEIKRTVAAPSVFIFPPS |
| | underlined) | DEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSFNRGEC |

1. A stable, pharmaceutical composition comprising an anti-CD45 antibody drug conjugate (ADC), a buffer, an antioxidant, a sugar, and a surfactant, wherein the ADC comprises a cytotoxin conjugated via a linker to an anti-CD45 antibody, wherein the anti-CD45 antibody comprises a heavy chain variable region comprising three heavy chain complimentary determining domains CDR1, CDR2, and CDR3 within the heavy chain variable region comprising the amino acid sequence set forth in SEQ ID NO: 1; and a light chain variable region comprising three light chain complimentary determining domains CDR1, CDR2, and CDR3 within the light chain variable region, wherein the light chain variable region comprising the amino acid sequence as set forth in SEQ ID NO:5.

2. The pharmaceutical composition of claim 1, wherein the antioxidant is sodium bisulfite, methionine, or both.

3. The pharmaceutical composition of claim 2, wherein the composition comprises at least one of:
a) about 25 to about 75 μM sodium bisulfite;
b) about 50 μM sodium bisulfite;
c) about 1-5 mM methionine; or
d) about 3 mM methionine.

4.-6. (canceled)

7. The pharmaceutical composition of claim 1, wherein the surfactant is polysorbate.

8. The pharmaceutical composition of claim 7, wherein the polysorbate is polysorbate 20; optionally about 0.01% polysorbate 20.

9. (canceled)

10. The pharmaceutical composition of claim 1, wherein the buffer is histidine.

11. The pharmaceutical composition of claim 10, wherein the composition comprises:
a) a concentration of histidine suitable to buffer the composition to a pH of about 5.8 to 6.2; or
b) about 5 to about 15 mM histidine.

12. (canceled)

13. The pharmaceutical composition of claim 1, wherein the sugar is sucrose; optionally about 10% sucrose.

14. (canceled)

15. A stable, pharmaceutical composition comprising an anti-CD45 antibody drug conjugate (ADC), histidine, sodium bisulfite, sucrose, methionine and polysorbate 20, wherein the ADC comprises a cytotoxin conjugated via a linker to an anti-CD45 antibody, wherein the anti-CD45 antibody comprises a heavy chain variable region comprising a CDR1 domain comprising the amino acid sequence as set forth in SEQ ID NO: 2, a CDR2 domain comprising the amino acid sequence as set forth in SEQ ID NO: 3, and a CDR3 domain comprising the amino acid sequence as set forth in SEQ ID NO: 4; and comprises a light chain variable region comprising a CDR1 domain comprising the amino acid sequence as set forth in SEQ ID NO: 6, a CDR2 domain comprising the amino acid sequence as set forth in SEQ ID NO: 7, and a CDR3 domain comprising the amino acid sequence as set forth in SEQ ID NO: 8.

16. The pharmaceutical composition of claim 15, wherein the composition comprises:
a) about 25 to about 75 μM sodium bisulfite, about 5 to about 15 mM histidine, about 5% to 10% sucrose, and about 5 to 15 mM methionine; or
b) about 10 mM histidine, about 50 uM sodium bisulfite, 10% sucrose, and 3 mM methionine.

17. The pharmaceutical composition of claim 16, comprising about 0.01% polysorbate 20.

18. (canceled)

19. The pharmaceutical composition of claim 1, wherein the pharmaceutical composition has a pH of about 5.8 to about 6.2; or wherein the pharmaceutical composition has a pH of about 6.

20. (canceled)

21. The pharmaceutical composition of claim 1, wherein the antibody is an IgG1.

22. The pharmaceutical composition of claim 1, wherein the anti-CD45 antibody is conjugated to the linker via a cysteine residue at amino acid position 265 of the Fc region (numbering is EU by Kabat).

23. The pharmaceutical composition of claim 1, wherein the anti-CD45 antibody comprises:
a) a heavy chain variable region amino acid sequence as set forth in SEQ ID NO: 1 and a light chain variable region amino acid sequence as set forth in SEQ ID NO: 5; or
b) a heavy chain amino acid sequence as set forth in SEQ ID NO: 9 and a light chain amino acid sequence as set forth in SEQ ID NO: 10.

24. (canceled)

25. The pharmaceutical composition of claim 1, wherein the pharmaceutical composition is liquid or lyophilized.

26. The pharmaceutical composition of claim 1, wherein the ADC has a toxin:antibody ratio of about 1.8 to 2.2; or wherein the ADC has a toxin:antibody ratio of 2.

27. (canceled)

28. The pharmaceutical composition of claim 1, wherein the cytotoxin is an indolinobenzodiazepine (IGN); optionally, wherein the IGN has a formula 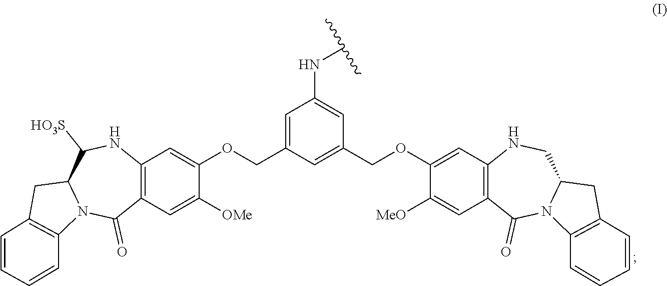
or the IGN has a formula 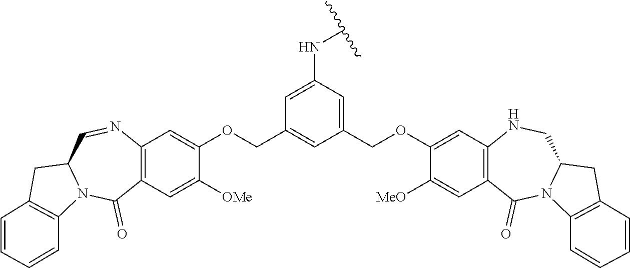 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]
wherein the wavy line indicates the point of covalent attachment to the linker of the ADC.

29. (canceled)

30. The pharmaceutical composition of claim 1, wherein the ADC has the formula

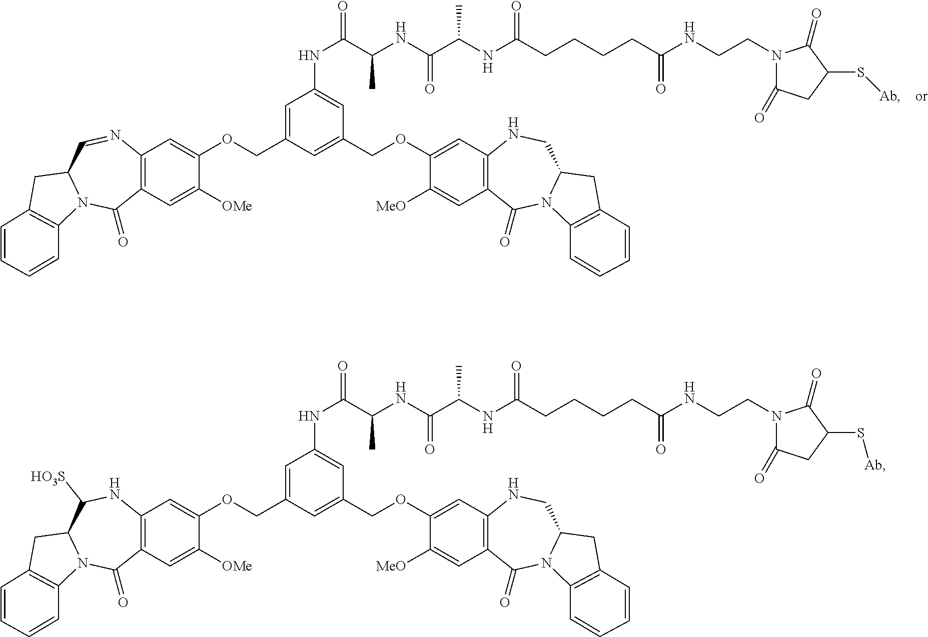

31.-48. (canceled)

49. The pharmaceutical composition of claim 1, wherein the anti-CD45 antibody comprises a heavy chain variable region amino acid sequence as set forth in SEQ ID NO: 1 and a light chain variable region amino acid sequence as set forth in SEQ ID NO: 5; or wherein the anti-CD45 antibody comprises a heavy chain amino acid sequence as set forth in SEQ ID NO: 9 and a light chain amino acid sequence as set forth in SEQ ID NO: 10.

50. (canceled)


SEQUENCE LISTING (72 sequences)

SEQ ID NO: 1 - protein, 127 aa, synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTMVTVSS

SEQ ID NO: 2 - protein, 9 aa, synthetic construct
FTFEAYSMN

SEQ ID NO: 3 - protein, 17 aa, synthetic construct
YISLSGATIHYADSVKG

SEQ ID NO: 4 - protein, 20 aa, synthetic construct
ARGGQYYYTSSDYGEVAFDI

SEQ ID NO: 5 - protein, 111 aa, Synthetic construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIK

SEQ ID NO: 6 - protein, 15 aa, synthetic construct
RSSQSLVSSGYNYLD

SEQ ID NO: 7 - protein, 7 aa, synthetic construct
FGSSRAS

SEQ ID NO: 8 - protein, 9 aa, Synthetic construct
MQRRRTPWS

SEQ ID NO: 9 - protein, 457 aa, Synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 10 - protein, 218 aa, synthetic construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1473 bp, synthetic construct
  source 1..1473: mol_type = other DNA
ggtaccgccaccatggatctgctgcacaagaacatgaagcacctgtggttctttctgctgctggtggccgctcctagatgggtgctgtctgaagtgcagctggtggaatctggcggaggattggttcagcctggcggctctctgagactgtcttgtgctgcctctggcttcaccttcgaggcctactccatgaactgggtccgacaggctcctggcaaaggactggaatgggtgtcctacatctctctgtctggcgccaccatccactacgccgattctgtgaagggcagattcaccatcagccgggacaacgccaagaactccctgtacctgcagatgaacagcctgagagccgaggacaccgccgtgtactattgtgctagaggcggccagtactactacacctcctctgattacggcgaggtggccttcgatatctggggccagggaacaatggtcaccgtgtcctctgcttccaccaagggaccttccgtgtttcctctggctccttccagcaagtctacctctggtggaaccgctgctctgggctgcctggtcaaggattactttcctgagcctgtgaccgtgtcttggaactctggtgctctgacctccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcggaccctctgtgttcctgtttccacctaagcctaaggacaccctgatgatctctcggacccctgaagtgacctgcgtggtcgtgtgtgtgtctcacgaggacccagaagtgaagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtttacacactgcctccatctcgggacgagctgaccaagaatcaggtgtccctgacctgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaacgcctacacacagaagtccctgtctctgtcccctggcaagtgatagaagctt

SEQ ID NO: 12 - DNA, 756 bp, synthetic construct
  source 1..756: mol_type = other DNA
ggatccgccaccatggatctgctgcacaagaacatgaagcacctgtggttctttctgctgctggtggccgctcctagatgggtgctgtctgacatcgtgatgacccagtctccactgagcctgcctgttacacctggcgagcctgccagcatctcctgcagatcttcccagtctctggtgtcctccggctacaactacctggactggtatctgcagaagcccggccagtctcctcagctgctgatctactttggctcctccagagcctctggcgtgcccgatagattttctggctctggctccggcaccgacttcaccctgaagatctctagagtggaagccgaggacgtgggcgtgtactactgtatgcagcggagaagaaccccttggtctttcggcggaggcaccaaggtggaaatcaagagaaccgtggctgccccttccgtgttcatcttcccaccatctgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgctgatagctcgag

SEQ ID NO: 13 - protein, 127 aa, synthetic construct
QVQLVESGGGLVKPGGSLRLSCAASGFTFSSYSMNWVRQAPGKGLEWVSYISSSSSTIYYADSVKGRFTISRDNAKNSLYLQMNSLRAEDTAVYYCARGGQYYYDSSRYGEVAFDIWGQGTMVTVSS

SEQ ID NO: 14 - protein, 9 aa, synthetic construct
FTFSSYSMN

SEQ ID NO: 15 - protein, 17 aa, synthetic construct
YISSSSSTIYYADSVKG

SEQ ID NO: 16 - protein, 20 aa, synthetic construct
ARGGQYYYDSSRYGEVAFDI

SEQ ID NO: 17 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQRRRTPPFTFGGGTKVEIK

SEQ ID NO: 18 - protein, 16 aa, synthetic construct
RSSQSLLHSNGYNYLD

SEQ ID NO: 19 - protein, 7 aa, synthetic construct
LGSNRAS

SEQ ID NO: 20 - protein, 10 aa, synthetic construct
MQRRRTPPFT

SEQ ID NO: 21 - protein, 457 aa, synthetic construct
QVQLVESGGGLVKPGGSLRLSCAASGFTFSSYSMNWVRQAPGKGLEWVSYISSSSSTIYYADSVKGRFTISRDNAKNSLYLQMNSLRAEDTAVYYCARGGQYYYDSSRYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 22 - protein, 220 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 127 aa, synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYDSSDYGEVAFDIWGQGTMVTVSS

SEQ ID NO: 24 - protein, 9 aa, synthetic construct
FTFEAYSMN

SEQ ID NO: 25 - protein, 17 aa, synthetic construct
YISLSGATIHYADSVKG

SEQ ID NO: 26 - protein, 20 aa, synthetic construct
ARGGQYYYDSSDYGEVAFDI

SEQ ID NO: 27 - protein, 111 aa, synthetic construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPWSFGGGTKVEIK

SEQ ID NO: 28 - protein, 15 aa, synthetic construct
RSSQSLVSNGYNYLD

SEQ ID NO: 29 - protein, 7 aa, synthetic construct
FGSSRAS

SEQ ID NO: 30 - protein, 9 aa, synthetic construct
MQRRRTPWS

SEQ ID NO: 31 - protein, 457 aa, synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYDSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 32 - protein, 218 aa, synthetic construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 127 aa, synthetic construct
QVQLVESGGGLVKPGGSLRLSCAASGFTFGGYSMNWVRQAPGKGLEWVSYISISGATITYADSVKGRFTISRDNAKNSLYLQMNSLRAEDTAVYYCARGGQYYYDSSDYGEVAFDIWGQGTMVTVSS

SEQ ID NO: 34 - protein, 9 aa, synthetic construct
FTFGGYSMN

SEQ ID NO: 35 - protein, 17 aa, synthetic construct
YISISGATITYADSVKG

SEQ ID NO: 36 - protein, 20 aa, synthetic construct
ARGGQYYYDSSDYGEVAFDI

SEQ ID NO: 37 - protein, 112 aa, synthetic construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPPFTFGGGTKVEIK

SEQ ID NO: 38 - protein, 15 aa, synthetic construct
RSSQSLVSNGYNYLD

SEQ ID NO: 39 - protein, 7 aa, synthetic construct
FGSSRAS

SEQ ID NO: 40 - protein, 10 aa, synthetic construct
MQRRRTPPFT

SEQ ID NO: 41 - protein, 457 aa, synthetic construct
QVQLVESGGGLVKPGGSLRLSCAASGFTFGGYSMNWVRQAPGKGLEWVSYISISGATITYADSVKGRFTISRDNAKNSLYLQMNSLRAEDTAVYYCARGGQYYYDSSDYGEVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 42 - protein, 219 aa, synthetic construct
DIVMTQSPLSLPVTPGEPASISCRSSQSLVSNGYNYLDWYLQKPGQSPQLLIYFGSSRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 127 aa, synthetic construct; identical to SEQ ID NO: 1

SEQ ID NO: 44 - protein, 9 aa, synthetic construct
FTFEAYSMN

SEQ ID NO: 45 - protein, 17 aa, synthetic construct
YISLSGATIHYADSVKG

SEQ ID NO: 46 - protein, 20 aa, synthetic construct
ARGGQYYYTSSDYGEVAFDI

SEQ ID NO: 47 - protein, 111 aa, synthetic construct; identical to SEQ ID NO: 27

SEQ ID NO: 48 - protein, 15 aa, synthetic construct
RSSQSLVSNGYNYLD

SEQ ID NO: 49 - protein, 7 aa, synthetic construct
FGSSRAS

SEQ ID NO: 50 - protein, 9 aa, synthetic construct
MQRRRTPWS

SEQ ID NO: 51 - protein, 457 aa, synthetic construct; identical to SEQ ID NO: 9

SEQ ID NO: 52 - protein, 218 aa, synthetic construct; identical to SEQ ID NO: 32

SEQ ID NO: 53 - protein, 127 aa, synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTLVTVSS

SEQ ID NO: 54 - protein, 9 aa, synthetic construct
FTFEAYSMN

SEQ ID NO: 55 - protein, 17 aa, synthetic construct
YISLSGATIHYADSVKG

SEQ ID NO: 56 - protein, 20 aa, synthetic construct
ARGGQYYYTSSDYGEVAFDI

SEQ ID NO: 57 - protein, 111 aa, synthetic construct
DIVLTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIK

SEQ ID NO: 58 - protein, 15 aa, synthetic construct
RSSQSLVSSGYNYLD

SEQ ID NO: 59 - protein, 7 aa, synthetic construct
FGSSRAS

SEQ ID NO: 60 - protein, 9 aa, synthetic construct
MQRRRTPWS

SEQ ID NO: 61 - protein, 457 aa, synthetic construct
EVQLVESGGGLVQPGGSLRLSCAASGFTFEAYSMNWVRQAPGKGLEWVSYISLSGATIHYADSVKGRFTISRDNAKNSLYLQMNSLRAEDTAVYYCARGGQYYYTSSDYGEVAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 62 - protein, 218 aa, synthetic construct
DIVLTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPWSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 127 aa, synthetic construct; identical to SEQ ID NO: 33

SEQ ID NO: 64 - protein, 9 aa, synthetic construct
FTFGGYSMN

SEQ ID NO: 65 - protein, 17 aa, synthetic construct
YISISGATITYADSVKG

SEQ ID NO: 66 - protein, 20 aa, synthetic construct
ARGGQYYYDSSDYGEVAFDI

SEQ ID NO: 67 - protein, 112 aa, synthetic construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPPFTFGGGTKVEIK

SEQ ID NO: 68 - protein, 15 aa, synthetic construct
RSSQSLVSSGYNYLD

SEQ ID NO: 69 - protein, 7 aa, synthetic construct
FGSSRAS

SEQ ID NO: 70 - protein, 10 aa, synthetic construct
MQRRRTPPFT

SEQ ID NO: 71 - protein, 457 aa, synthetic construct; identical to SEQ ID NO: 41

SEQ ID NO: 72 - protein, 219 aa, synthetic construct
DIVMTQSPLSLPVTPGEPASISCRSSQSLVSSGYNYLDWYLQKPGQSPQLLIYFGSSRASGVPDRFSGSGSGTDFTLKISRVEAEDVGVYYCMQRRRTPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC